(19) United States
(12) Patent Application Publication
(10) Pub. No.: US 2020/0237615 A1
(43) Pub. Date: Jul. 30, 2020

(54) STOCHASTIC STIMULATION TO IMPROVE INFANT RESPIRATION

(71) Applicants: PRESIDENT AND FELLOWS OF HARVARD COLLEGE, Cambridge, MA (US); UNIVERSITY OF MASSACHUSETTS, Boston, MA (US)

(72) Inventors: James B. NIEMI, Concord, MA (US); David PAYDARFAR, Austin, TX (US)

(73) Assignees: PRESIDENT AND FELLOWS OF HARVARD COLLEGE, Cambridge, MA (US); UNIVERSITY OF MASSACHUSETTS, Boston, MA (US)

(21) Appl. No.: 16/639,274

(22) PCT Filed: Aug. 14, 2018
(86) PCT No.: PCT/US2018/046603

§ 371 Date: Feb. 14, 2020

Related U.S. Application Data
(60) Provisional application No. 62/546,401, filed on Aug. 16, 2017.

(51) Int. Cl. A61H 31/00; A61B 5/0205; A61B 5/021; A61B 5/08; A61B 5/00; A61B 5/145

(52) U.S. Cl. CPC A61H 31/00; A61H 2201/5007; A61B 5/02108; A61B 5/0816; A61B 5/4818; A61B 5/7264; A61B 5/725; A61B 5/726; A61B 5/14542; A61B 2503/04; A61H 2201/0146; A61H 2201/0173; A61H 2201/1207; A61H 2201/1619; A61H 2201/5058; A61B 5/0205

(57) ABSTRACT

The inventors have developed systems and methods for providing stochastic stimulus to patients to improve their respiration. For instance, the inventors have discovered that ventilated infants improve their breathing including by reducing the total amount of desaturation during periods of stochastic, mechanical stimulation. It was previously thought that stochastic stimulation only improved breathing by encouraging active pacemaker activity. Accordingly, the inventors have developed systems and methods that improves infant respiration.

### CROSS REFERENCE TO RELATED APPLICATIONS

This application claims priority to U.S. Patent Application No. 62/546,401, filed Aug. 16, 2017, titled “Stochastic Stimulation to Improve Infant Respiration,” the contents of which are incorporated herein by reference.

### STATEMENT REGARDING FEDERALLY SPONSORED RESEARCH

This invention was made with government support under Grant No. U.S. Pat. No. 1,401,711 awarded by the National Science Foundation. The government has certain rights in the invention.

### TECHNICAL FIELD

The present invention relates to methods and systems for improving infant respiration using stochastic stimulation.

### BACKGROUND

Infants commonly have irregular breathing patterns with periodic and sporadic pauses in breathing, more commonly referred to as “apnea.” One way to analyze breathing patterns is use the time interval between breaths, also referred to as the “interbreath interval” or “IBI.”

Preterm infant breathing patterns are highly irregular, with rapid changes in measures of breathing. Standard statistical measures such as mean and variance of the interbreath interval have been used in an attempt to quantify the variability of breathing in preterm infants. However, the sole measurement of interbreath intervals does not reveal the full magnitude of the issue. Spontaneously breathing premature, and some mature, infants suffer apneic events resulting in dangerous low oxygenation episodes, hypoxia, as well as drops in heart rate, bradycardia. It is known that ventilator dependent infants also suffer from periods of hypoxia and bradycardia, even if their breathing patterns, and thereby interbreath intervals, are stabilized by the ventilator.

It is believed that apneic events and poor respiratory function may also be contributing factors to Sudden Infant Death Syndrome. Even if not fatal, it is believed that apneic events and poor respiratory function may have a number of adverse consequences such as lengthening hospital stays, delaying the cognitive and executive development of an infant, or even irreparably harming the infant. These apneic events during infancy may affect the individual for their entire lifespan.

### SUMMARY

The inventors have developed systems and methods for providing stochastic stimulus to patients to improve their respiration. For instance, the inventors have discovered that ventilated infants improve their breathing including by reducing the total amount of desaturation during periods of stochastic, mechanical stimulation. It was previously thought that stochastic stimulation only improved breathing by encouraging active pacemaker activity.

Particularly, the inventors have performed a clinical study that tested the hypothesis that stochastic stimulation could provide additional benefits beyond the encouragement of breathing pacemaker neuron drive. During the study, ventilator dependent infants not dependent on pacemaker drive were placed on the mattress with 30 minute on/off cycles. Ventilator dependent infants are an interesting population because they can suffer hypoxia and oxygen instability episodes even though their breathing rates are stabilized by a machine. In a preliminary analysis, the stimulation decreased the duration of the hypoxia by 30% (p=0.04) and decreased the variance in oxygenation (SaO2) by 20% (p=0.025) when compared to the non-stimulation period. This is a novel finding since the previous effect was thought to be purely caused by encouraging the pacemaker drive.

According to another embodiment, a method for improving a patient's respiration by decreasing the total amount of oxygen desaturation time. In other examples, disclosed is a method of preventing an apneic or hypoxic event that includes the acts of receiving physiological data from a subject, analyzing the received physiological data to detect at least one of an impending apneic event or an impending hypoxic event, and applying a stimulation to the subject to inhibit occurrence of the impending apneic or the impending hypoxic event. The physiological data includes respiratory data. The analyzing includes use of a point-process model and gross body movement data of the subject. The stimulating occurs after an occurrence of a predetermined condition.

According to another embodiment, a method for inhibiting an occurrence of an apneic or hypoxic event includes the acts of receiving physiological data from a subject, analyzing the received physiological data to detect at least one of an impending apneic event or an impending hypoxic event, and applying a stimulation to the subject to inhibit occurrence of the impending apneic or the impending hypoxic event. The physiological data includes circulatory data. The analyzing includes use of a point-process model and gross body movement data of the subject. The stimulating occurs after an occurrence of a predetermined condition.

According to yet another embodiment, a system for inhibiting an apneic event or a hypoxic event includes an analysis module and a stimulating mechanism. The analysis module is configured to receive physiological data from a subject and to analyze the received physiological data in real time. The physiological data includes cardiological data. The analysis includes using a point-process model to detect at least one of an impending apneic event or an impending hypoxic event and further includes use of a point-process model and gross body movement data of the subject. The stimulating mechanism is operatively coupled to the analysis module. The stimulating mechanism is configured to apply a stimulus to the subject. The applied stimulus inhibits the impending apneic event or the impending hypoxic event.

According to yet another embodiment, a system for inhibiting an apneic event or a hypoxic event includes an analysis module and a stimulating mechanism. The analysis module is configured to receive physiological data from a subject and to analyze the received physiological data in real time. The physiological data includes respiratory data. The analysis includes using a point-process model to detect at least one of an impending apneic event or an impending hypoxic event and further includes use of a point-process model and gross body movement data of the subject. The stimulating mechanism is operatively coupled to the analysis module. The stimulating mechanism is configured to apply a stimulus to the subject. The applied stimulus inhibits the impending apneic event or the impending hypoxic event.

According to yet another embodiment, a system can improve the respiratory function of a patient and the system can include a pressure support system and a controller. The pressure support system can be configured to provide breathable air to the patient. The controller can be configured to receive a mechanical stimulation signal. The controller can then provide for determining an altered signal based on deriving a set of parameters from the received mechanical stimulation signal. The controller can then adjust an air pressure of the pressure support system based on the altered signal. In some examples, the mechanical stimulation signal can be automatically adjusted based on a measurement of respiratory function used to predict an upcoming respiratory state. In some examples, determining the altered signal can further comprise accounting for an optimal oxygen saturation of the patient. In some examples, the adjusting can be coordinated with the mechanical stimulation signal.

Additional aspects of the invention will be apparent to those of ordinary skill in the art in view of the detailed description of various embodiments, which is made with reference to the drawings, a brief description of which is provided below.

### BRIEF DESCRIPTION OF THE FIGURES

FIG. 1A illustrates the interbreath interval of simulated data.

FIG. 1B is an instantaneous variance estimated by a point process model using the data of FIG. 1A.

FIG. 2 shows a Kolmogorov-Smirnov plot of time-rescaled quantiles derived from the simulated data of FIG. 1A.

FIG. 3A is an example from one continuous recording of a newborn rat.

FIG. 3B is a calculated variance of the data in FIG. 3A using the point process model.

FIG. 4A shows a Kolmogorov-Smirnov plot of time-rescaled quantiles derived for data of a newborn rat.

FIG. 4B shows an autocorrelation plot for the newborn rat data of FIG. 4A.

FIG. 4C shows a Kolmogorov-Smirnov plot of time-rescaled quantiles derived for data of a second newborn rat.

FIG. 4D shows an autocorrelation plot for the second newborn rat data of FIG. 4C.

FIG. 5A shows one continuous recording of a human infant interbreath interval.

FIG. 5B shows the calculated variance of the data in FIG. 5A using the point process algorithm.

FIG. 6A shows the Kolmogorov-Smirnov plot of the infant data of FIG. 5A.

FIG. 6B shows the Kolmogorov-Smirnov plot of a second infant data.

FIG. 6C shows the Kolmogorov-Smirnov plot of a third infant data.

FIG. 6D shows the Kolmogorov-Smirnov plot of a fourth infant data.

FIG. 7A shows an example of interbreath interval variance over time when stimulation was initiated.

FIG. 7B shows an example of interbreath interval variance over time when stimulation was terminated.

FIG. 8A shows a flowchart for an algorithm 700 to monitor physiological instabilities in real time.

FIG. 8B shows a system to monitor instabilities in breathing over time and control stimulation according to one embodiment.

FIG. 9 depicts the cross-section of a therapeutic mattress design that applies isolated stochastic resonance mechanostimulation to a portion of the mattress according to one embodiment.

FIG. 10 shows an exploded view of an active assembly according to one embodiment.

FIG. 11 shows results from the test of the single-bodied mattress compared to the isolation mattress of FIG. 9.

FIG. 12 shows a graph of mattress output for the isolation mattress, comparing the output of the active and passive regions.

FIG. 13 depicts measurement locations in one embodiment used for the mattress displacement tests.

FIG. 14 shows a system for focal stimulation according to one embodiment.

FIG. 15A shows a support structure or garment according to one embodiment.

FIG. 15B shows a support structure or garment according to another embodiment.

FIG. 16A depicts stimulation array according to one embodiment.

FIG. 16B depicts single piece of the stimulation array.

FIG. 17 shows graph of an example pulse plethysmograph signal and a pulse-plethysmograph-derived gross body movement amplitude signal.

FIG. 18 illustrates a receiver operating characteristic curve shown from the prediction scores of all sip patients used in an example study

FIG. 19 demonstrates the relationship between IBI and the movement signal derived from the discrete plethysmograph signal.

FIG. 20 shows a graph of the condition protocol for each infant through a study.

FIG. 21 depicts a graph showing an example of improvement in a single infant over one hour where the condition has been changed from stimulus ON to stimulus OFF.

FIG. 22 depicts a graph showing the effect of therapeutic stimulation may be reduced for very low birth weight infants prompting the consideration of adjustments in stimulation based on infant mass.

FIG. 23 shows an exemplary artificial respiratory support apparatus, according to an embodiment of the present disclosure.

### DETAILED DESCRIPTION OF PREFERRED EMBODIMENTS

Unless defined otherwise, technical and scientific terms used herein have the same meaning as commonly understood by one of ordinary skill in the art to which this invention belongs. Szycher's Dictionary of Medical Devices CRC Press, 1995, may provide useful guidance to many of the terms and phrases used herein. One skilled in the art will recognize many methods and materials similar or equivalent to those described herein, which could be used in the practice of the present invention. Indeed, the present invention is in no way limited to the methods and materials specifically described.

In some embodiments, properties such as dimensions, shapes, relative positions, and so forth, used to describe and claim certain embodiments of the invention are to be understood as being modified by the term “about.”

Various examples of the invention will now be described. The following description provides specific details for a thorough understanding and enabling description of these examples. One skilled in the relevant art will understand, however, that the invention may be practiced without many of these details. Likewise, one skilled in the relevant art will also understand that the invention can include many other obvious features not described in detail herein. Additionally, some well-known structures or functions may not be shown or described in detail below, so as to avoid unnecessarily obscuring the relevant description.

The terminology used below is to be interpreted in its broadest reasonable manner, even though it is being used in conjunction with a detailed description of certain specific examples of the invention. Indeed, certain terms may even be emphasized below; however, any terminology intended to be interpreted in any restricted manner will be overtly and specifically defined as such in this Detailed Description section.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations may be depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

### Stochastic Stimulation: Improving Respiratory Function Generally

The inventors have developed systems and methods for providing stochastic stimulus to patients to improve their respiration. For instance, the inventors have discovered that ventilated infants improve their breathing by reducing the total amount of desaturation during periods of stochastic, mechanical stimulation. It was previously thought that stochastic stimulation only improved breathing by encouraging active pacemaker activity. Accordingly, the inventors have developed systems and methods that improves infant respiration.

Particularly, the inventors have performed a clinical study that tested the hypothesis that stochastic stimulation could provide additional benefits beyond the encouragement of breathing pacemaker neuron drive. During the study, ventilator dependent infants were placed on the mattress with 30 minute on/off cycles of stimulation. Ventilator dependent infants are an interesting population because they can suffer hypoxia and oxygen instability episodes even though their breathing rates are stabilized by a machine. In a preliminary analysis, the stimulation decreased the duration of the hypoxia by 30% (p=0.04) and decreased the variance in oxygenation (SaO2) by 20% (p=0.025) when compared to the non-stimulation period. This is a novel finding since the previous effect was thought to be purely caused by encouraging the pacemaker drive.

Accordingly, the inventors have developed systems and methods to deliver stochastic stimulation to patients for scenarios other than just to address life-threatening breathing events. Particularly, the inventors have discovered that infants and other patients may benefit from continuous or periodic stochastic stimulation that decreases the overall level of hypoxia—rather than just protecting against dangerous hypoxia events.

The periodic or continuous stimulation may yield long term benefits to patients, including improved cognitive function among a host of other advantages. Furthermore, the inventors have discovered that the amount of benefit of stochastic stimulation at a certain frequency and intensity appear to vary with the weight of infants. For instance, in a study performed by the inventors, the lightest infants had the lowest amount of respiratory improvement from the specific frequency and intensity of stochastic stimulation provided. Accordingly, the inventors have developed systems and methods that increase or decrease the frequency and/or intensity of the stochastic stimulation based on the weight of the patient.

In some examples, a system may adjust the frequency of stochastic stimulation based on feedback from an oxygenation or other sensor(s) to optimize breathing for each individual patient. Accordingly, other individual factors that may play a role in the effectiveness of the stimulation (e.g., age, compliance of lung tissue) may also be accounted for to optimize the stimulation levels.

### Stochastic Stimulation: Life Threatening Event Avoidance

A point-process modeling framework may be used to develop algorithms for detecting and predicting life-threatening events in neonates. These life-threatening events include apnea, bradycardia, and hypoxia. A number of physiological signals may be monitored to automatically detect, and even predict the occurrence of life-threatening events. Detection or prediction of these events may decrease the severity of an event or even completely eliminate the event. Once detected, methods and systems may automatically apply a stimulus to a subject to decrease the severity of the event, revert the subject to the normal, rhythmic state, or even entirely prevent the occurrence of the event.

The application of stochastic resonance to non-linear physiologic systems may improve system performance. For example, the application of stochastic noise via mechanical vibration enhances the respiratory performance of infants with apnea and hypoxia. Additionally, it may be the case that stochastic resonance might also improve the pulmonary system's ability to optimize oxygen tension and gas exchange.

### Modeling of Interbreath Intervals

Respiratory rhythm in mammals is governed by neural circuits within the brainstem that signal the timing and depth of each breath. Continuous ventilation results from recurrent bursts of inspiratory neuronal activity that controls the diaphragm via discrete phrenic motor neuron activations. One assumption that allows non-invasive measurement of neuronal inspiratory bursts is to assume that the peak of inspiration is a discrete event that marks the timing of neuronal inspiratory bursts. Another assumption that may be made is that interbreath interval dynamics are governed by continuous processes under the regulation of multiple feedback and feed-forward loops impinging upon the respiratory oscillator.

The interbreath interval of an infant follows a power-law distribution. The characterizing parameters of the power-law distribution are found to be sensitive to age (e.g., maturation). During a respiratory cycle, the end of inspiration and onset of expiration mark local maxima or local minima. For the purposes of this disclosure, the end of inspiration and onset of expiration will define local maxima unless otherwise noted. In an observation interval (0, T], the times where the local maxima occur may be defined as 0<u₁<u₂<Λ<uₖ<Λ<u_{K}≤T. Then, for any given respiratory event uₖ, the waiting time until the next event obeys a history dependent log-normal probability density f(t|Hₖ,θ) as

$$f ( t \vert {H}_{k} , θ ) = {[ \frac{1}{2 {{πσ}^{2} ( t - {u}_{k} )}^{2}} ]}^{\frac{1}{2}} exp \{ - \frac{1}{2} \frac{{( ln ( t - {u}_{k} ) - μ ( {H}_{k} , θ ) )}^{2}}{2 {σ}^{2}} \} \qquad ( 1 )$$

Time t is any time greater than uₖ. Hₖ is the history of interbreath intervals up to uₖ represented as Hₖ={uₖ,wₖ,wₖ₋₁,Λ,wₖ₋ₚ₊₁} where wₖ is the k^{th} interbreath interval represented as wₖ=uₖ−uₖ₋₁. Theta (θ) is a vector of model parameters. The instantaneous mean is modeled as a p-order autoregressive process,

$$μ ( {H}_{k} , θ ) = {θ}_{o} + {\sum }_{j = 1}^{p} {θ}_{j} {w}_{k - j + 1} .$$

The probability density in equation (1) defines the interbreath interval distribution with μ and σ as the characterizing parameters. The local maximum-likelihood approach is employed to estimate θ and σ at each instant of time t.

The local joint probability density of uₜ₋ₗ: uₜ is used to calculate the local maximum likelihood estimate of θ and σ where l is the length of the local likelihood observation interval. If a number nₜ of peaks in this interval are observed as u₁<u₂<Λ<uₙ_{ₜ}≤t and if θ as well as σ are time varying, then at time t, the maximum likelihood estimate of \hat{θ}ₜ and \hat{σ}ₜ is to be the estimate of θ and σ in the interval l. Considering the right censoring, the local log likelihood is obtained as

$$log f ( {u}_{t - l : t} \vert {θ}_{t} ) = {\sum }_{i = 2}^{{n}_{t}} w ( t - {u}_{i} ) log f ( {u}_{i} - {u}_{i - 1} \vert {H}_{{u}_{i - 1}} , {θ}_{t} ) + w ( t - {u}_{{n}_{t}} ) log {\int }_{t - {u}_{{x}_{t}}}^{∞} f ( θ \vert {H}_{{u}_{{n}_{t}}} , {θ}_{t} ) d θ \qquad ( 2 )$$

where w(t) is a weighting function to account for faster updates to local likelihood estimation. The weighing function is w(t)=exp(−α(t−u)) where α is the weighting time constant that assigns the influence of a previous observation on the local likelihood at time t. The instantaneous estimate of the mean μ may be obtained using the autoregressive representation because θ can be estimated in continuous time. Similarly, the local likelihood estimate provides the instantaneous estimate of variance σ².

The interbreath interval probability model along with the local maximum likelihood method provides an approach for estimating the instantaneous mean and instantaneous variance of the interbreath interval. These measures provide information about the changes in the characteristics of the distribution and information related to the irregularity of breathing. The time-rescaled interbreath interval was computed to obtain a goodness-of-fit measure. The time-rescaled interbreath interval is defined as:

τₖ=∫ᵤ_{ₖ₋ₜ}^{u}^{ₖ}λ(t|Hₜ,\hat{θ}ₜ)dt (3)

where uₖ represents the breathing events observed in (0,T) and λ(t|Hₜ,\hat{θ}ₜ) is the conditional intensity function defined as:

$$λ ( t \vert {H}_{t} , {\hat{θ}}_{t} ) = {f ( t \vert {H}_{t} , {\hat{θ}}_{t} , {\hat{σ}}_{t} ) [ 1 - {\int }_{{u}_{{n}_{t}}}^{t} f ( θ \vert {H}_{θ} , {\hat{θ}}_{θ} , {\hat{σ}}_{θ} ) d θ ]}^{- 1} \qquad ( 4 )$$

The conditional intensity is the history dependent rate function for a point process that generalizes the rate function for a Poisson process. The τₖ values are independent, exponential, random variables with a unit rate. With a transformation zₖ=1−exp(−τₖ), the zₖ values become independent, uniform random variables on the interval (0,1]. A Kolmogorov-Smirnov test was used to assess the agreement between the transformed zₖ values and a uniform probability density. A Kolmogorov-Smirnov plot indicates agreement of the point-process model with the interbreath interval data series by plotting the transformed zₖ values versus the uniform density. A line close to the 45 degree diagonal from this plot indicates close agreement.

The Kolmogorov-Smirnov distance measures the largest distance between the cumulative distribution function of the transformed interbreath interval and the cumulative distribution function of a uniform distribution, both on the interval (0,1]. A shorter Kolmogorov-Smirnov distance indicates a better model in terms of goodness-of-fit.

### EXAMPLES

The following examples are provided to better illustrate the claimed invention and are not intended to be interpreted as limiting the scope of the invention. To the extent that specific materials or steps are mentioned, it is merely for purposes of illustration and is not intended to limit the invention. One skilled in the art may develop equivalent means or reactants without the exercise of inventive capacity and without departing from the scope of the invention.

Data were analyzed from both human and animal trials. Neonatal rats exhibit respiratory patterns and chemo-responses analogous to preterm infants. This includes both periodically occurring apnea episodes and sporadic apneas with bradycardia and hypoxemia. One- to two-day-old rats were placed in a sealed chamber and breathed through a face mask and pneumotachogram. Respiratory airflow was recorded through the mask. Pressure within the plethysmographically sealed chamber was measured and these measurements were used as an index of respiratory effort.

The tested preterm infant data included infants having a gestational age of less than 36 weeks and post-conceptional age greater than 30 weeks at the time of study. The infants were spontaneously breathing room air or receiving supplemental oxygen through nasal cannulae at a fixed flow rate. Respiratory inductance plethysmography of abdominal movements during spontaneous breathing (Somnostar PT; Viasys Healthcare, Yorbalinda, Calif.) was used to collect respiratory signal data at a sampling rate of 100 Hz.

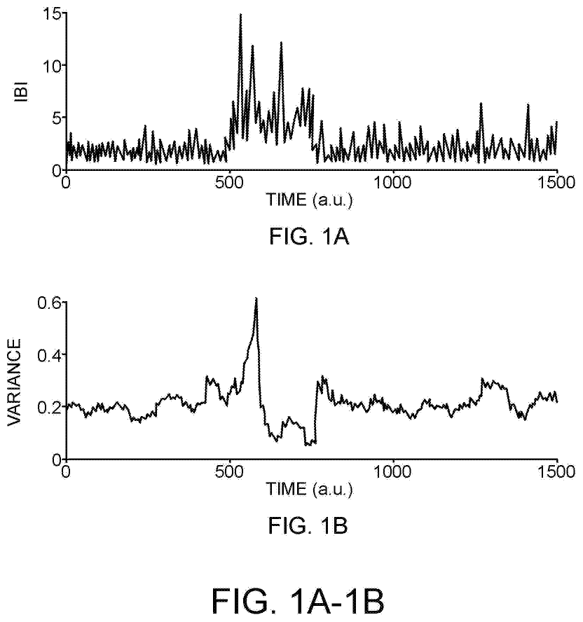

The model was first tested using simulated data sets. Interbreath interval data series were simulated from a log-normal distribution with set mean μ and variance σ² values. FIG. 1A illustrates one of the simulated data series. The interbreath interval (IBI) of the simulated data is plotted over time, which is shown with arbitrary units. The simulated data kept the interbreath intervals relatively stable between times of zero and 500 units. Then, the interbreath intervals experienced significant variance between times of 500 to 800 units. After the time of 800 units, the simulated interbreath intervals returned to same levels as between times of zero and 500 units. These data were generated by keeping the interbreath interval variance σ₂ at a fixed value for times zero to 500, then randomly altering the variance σ² for times 500 to 800, and then returning to the initial variance σ² for times greater than 800. The mean value μ was kept at a constant level. That is, times zero to 500 and times greater than 800 simulated non-apneic sleep and times 500 to 800 simulated the occurrence of apneic events.

Referring to FIG. 1B, the instantaneous variance estimated by the developed point process model of order p=4, with local likelihood window i=100 and weighting time constant α=0.01 along with a time resolution s=0.01 is shown for the data of FIG. 1A. As shown in FIG. 1B, the variance remained relatively constant at about 0.2 from times zero to 500, then sharply increased to about 0.6 and sharply fell to about 0.1 for times 500 to 800 before returning to about 0.2 for times after 800. This accurately estimated the mean μ and variance σ² for selected fixed mean μ and variance σ² values.

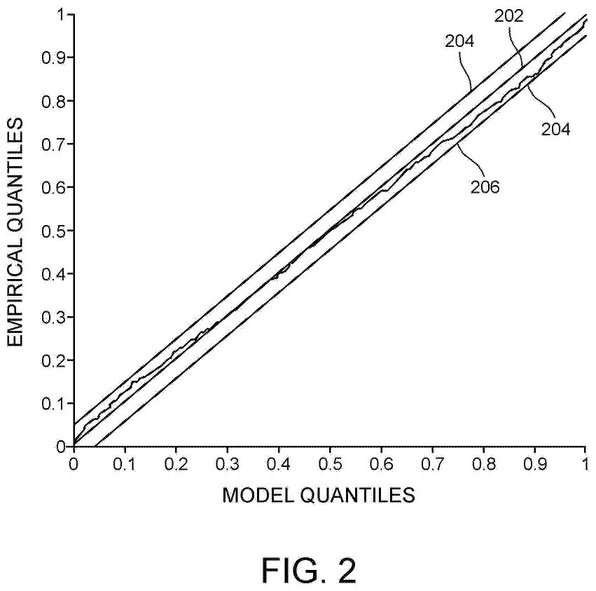

The goodness-of-fit of the point process model was analyzed. FIG. 2 shows a Kolmogorov-Smirnov plot of time-rescaled quantiles derived for the simulated data of FIG. 1A. The 95% confidence intervals 202 and theoretical values 204 were plotted along with the time-rescaled quantiles 206. A model is considered perfect if the quantiles 206 perfectly overlie the theoretical values 204. As shown, the time-rescaled quantiles 206 closely followed the line of theoretical values 204 and remain within the 95% confidence intervals 202.

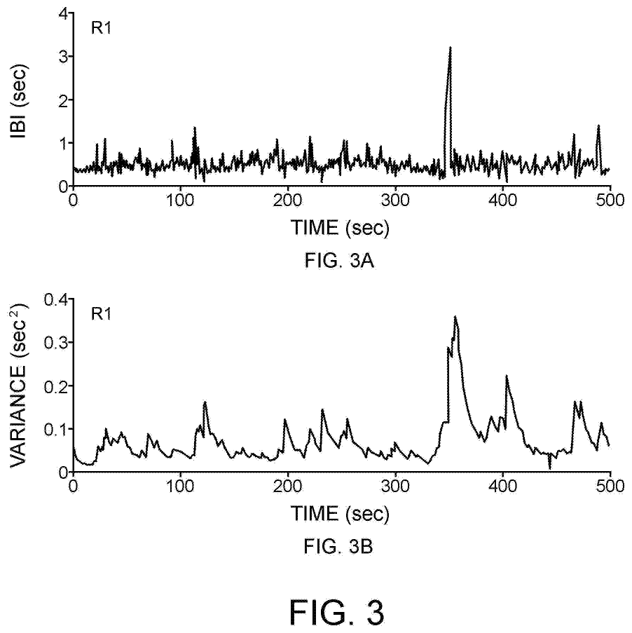

Referring to FIG. 3A, an example from one continuous recording of a newborn rat R1 is shown. FIG. 3A plots the interbreath interval (IBI) over time. As shown, the interbreath interval remained relatively stable and exceeded 1 second at relatively few points. In newborn rats, an interbreath interval greater than 1 second indicates apnea. Some peaks that exceed 1 second occurred at times of, for example, about 25 seconds, about 105 seconds, about 225 seconds, about 350 seconds, and about 490 seconds. As the apnea occurs, the variance increases.

The variance in the interbreath interval is an indicator of stability of breathing. FIG. 3B shows the calculated variance of the newborn rat R1 data in FIG. 3A using the point process algorithm. As shown, the variance remained relatively stable, with significant peaks formed at, for example, about 25 seconds, about 105 seconds, about 200, about 225 seconds, about 350 seconds, about 400 seconds, and about 490 seconds. These peaks correspond with the apneic interbreath interval peaks in FIG. 3A.

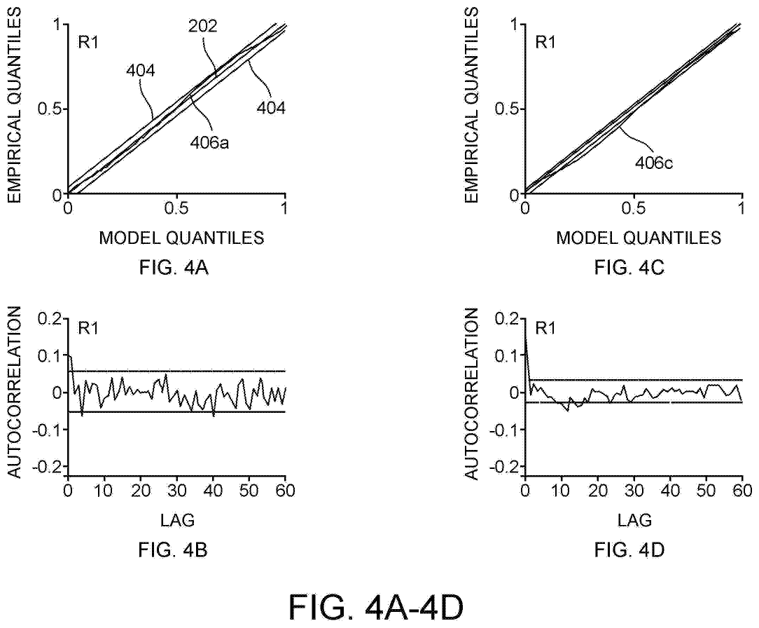

Referring to FIGS. 4A-4D, Kolmogorov-Smirnov plots of time-rescaled quantiles derived for data of two newborn rats R1, R2 was plotted along with the associated autocorrelation function for each. The theoretical values 402, 95% confidence intervals 404, and time-rescaled quantiles 406a,c for each newborn rat R1, R2 are shown in FIGS. 4A and 4C. The first newborn rat R1 was the same data used in FIGS. 3A and 3B. FIG. 4A shows the time-rescaled quantile 406a for the first newborn rat R1 closely following the theoretical values 402 along the 45 degree line, but approaching the upper 95% confidence interval 404 for model values between about 0.6 and about 0.8. FIG. 4B shows the autocorrelation of the first newborn rat R1 to remain within the corresponding confidence interval of (about ±0.05). FIG. 4C shows the time-rescaled quantile 406c for the second newborn rat R2 following the theoretical values 402 along the 45 degree line with a slight excursion beyond the lower 95% confidence interval 404 for model values between about 0.2 and about 0.4. FIG. 4D shows the autocorrelation of the second newborn rat R2 to remain within the corresponding confidence interval (about ±0.02).

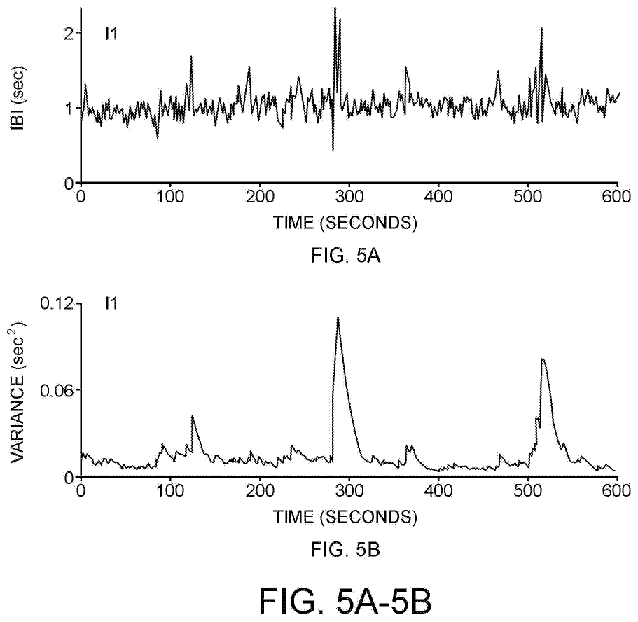

Referring to FIG. 5A, an example from one continuous recording of a human infant I1 is shown. The infant's I1 interbreath interval (IBI) remained at about one second peaks exceeding about 1.5 seconds at times of about 125 seconds, about 290, about 300 seconds, and about 510 seconds. In infants, the normal interbreath interval is about 1 second. Irregularity in breathing results in the interbreath interval varying from about 1 second to about 20 seconds. The change in interbreath interval is reflected as the variance.

FIG. 5B shows the variance of the interbreath interval data (FIG. 5A) of the infant I1. The instantaneous variance increased during the apnea, suggesting larger variability. The variance remained relatively steady at approximately 0.01 sec². Significant peaks were seen at times of about 125 seconds, about 290 to 300 seconds, and about 510 seconds.

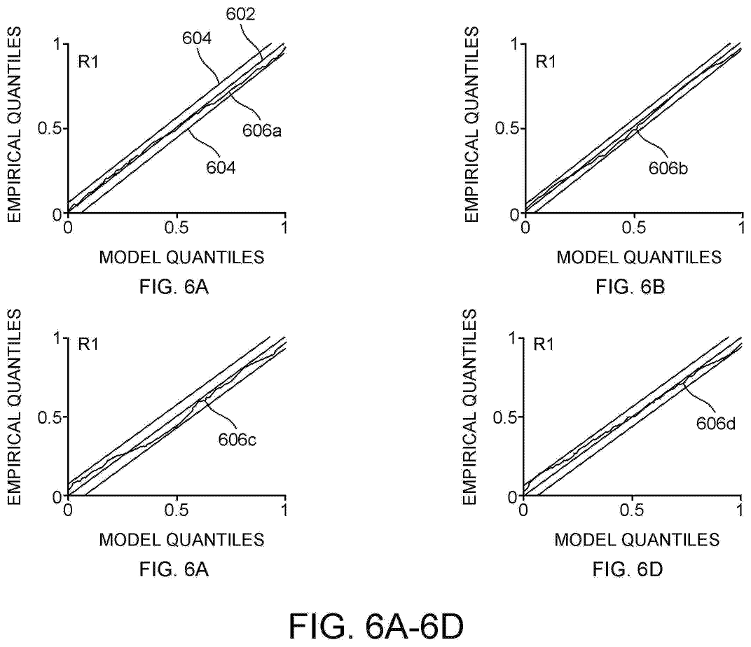

FIGS. 6A-6D provide the Kolmogorov-Smirnov plots from four infants I1-I4, respectively. FIG. 6A shows the Kolmogorov-Smirnov plot of the first infant I1 data from FIGS. 5A and 5B. The time-rescaled quantiles 606a for the first infant I1 closely track the theoretical values 602 along the 45 degree line, but approached the lower 95% confidence interval 604 for model quantiles of about 0.8 to about 1.0.

FIG. 6B shows the Kolmogorov-Smirnov plot of a second infant I2 data. The time-rescaled quantiles 606b for the second infant I2 closely tracked the theoretical values 602 along the 45 degree line, but approached the lower 95% confidence interval 604 for model quantiles of about 0.9 to about 1.0.

FIG. 6C shows the Kolmogorov-Smirnov plot of a third infant I3 data. The time-rescaled quantiles 606c for the third infant I3 tracked the theoretical values 602 along the 45 degree line. The time-rescaled quantiles 606c approached the upper 95% confidence interval 604 for model quantiles of about 0 to about 0.2 and approached the lower 95% confidence interval 604 for model quantiles of about 0.4 to about 0.6 and about 0.9 to about 1.0.

FIG. 6D shows the Kolmogorov-Smirnov plot of a fourth infant I4 data. The time-rescaled quantiles 606d for the fourth infant I4 closely tracked the theoretical values 602 along the 45 degree line. The time-rescaled quantiles 606d approached the upper 95% confidence interval 604 for model quantiles of about 0.1 to about 0.2 and approached lower 95% confidence interval 604 for model quantiles of about 0.9 to about 1.0.

The time varying evolution of the characterizing parameters were estimated to represent the dynamic nature of breathing and thereby provide a time-varying measure of irregularity in breathing according to Equation 1 above.

The instantaneous mean is modeled as a p-order autoregressive process as

$$μ ( {H}_{k} , θ ) = {θ}_{o} + {\sum }_{j = 1}^{p} {θ}_{j} {w}_{k - j + 1} \qquad ( 5 )$$

The probability density in Equation 1 defines the interbreath interval distribution with mean μ and variance σ as the characterizing parameters. At each instant of time t, a local maximum-likelihood approach was used to estimate μ and σ. To calculate the local maximum likelihood estimate of μ and σ, the local joint probability density of uₜ₋ₗ:uₜ l is defined as the length of the local likelihood observation interval. The maximum likelihood estimate of \hat{θ}ₜ and \hat{σ}ₜ is approximated as the estimate of θ and σ in the interval l at time t if nₜ peaks are observed within this interval as u₁<u₂<Λ<uₙ_{ₜ}≤t and if θ as well as σ are time varying. Thus, for a p-order of 4, Equation 1 becomes:

$$f ( t \vert {H}_{k} , θ ) = {[ \frac{1}{2 {πσ}^{2} {w}_{k}} ]}^{\frac{n - 2}{2}} exp \{ - \frac{1}{2} \frac{{( ln ( {w}_{k} ) - μ ( {H}_{k} , θ ) )}^{2}}{2 {σ}^{2}} \} \qquad ( 6 )$$

The order, p, can be set to a different level based on a particular application.

Given Eq. (6), the local log-likelihood for an observation window nₜ can be defined as:

$$log f ( {u}_{t - l : t} \vert {θ}_{t} ) = {\sum }_{i = 2}^{{n}_{t}} w ( t - {u}_{i} ) log f ( {u}_{i} - {u}_{i - 1} \vert {H}_{{u}_{i - 1}} , {θ}_{t} ) + w ( t - {u}_{{n}_{t}} ) log {\int }_{t - {u}_{{n}_{t}}}^{∞} f ( ϑ \vert {H}_{{u}_{{n}_{t}}} , {θ}_{t} ) d ϑ \qquad ( 7 )$$

where w(t) is a weighting function to account for faster updates to local likelihood estimation. The weighing function was expressed as w(t)=exp(−α(t−u)) where α is the weighting time constant that assigns the influence of a previous observation on the local likelihood at time t. The instantaneous estimate of the mean μ is obtained using the autoregressive representation because θ can be estimated in continuous time. Similarly, the local likelihood estimate provides the instantaneous estimate of variance σ² as

σ²=(ln(wₖ)−μₖ)²/nₜ (8)

Thus the instantaneous mean in Equation 5, along with the variance in Equation 8 determines the characterizing parameters of the algorithm that track the instability of breathing in real time.

### Modeling of Heartbeat Intervals

Additionally, or alternatively, other physiological signals can be monitored to detect or predict the occurrence of a life-threatening event. A point-process model was developed using electrocardiograph and respiratory signals as primary signals. All other physiological signals were used as covariates in the predictive algorithm.

The peak of the electrocardiogram, also known as the R-wave event, is treated as a point process. The distribution of the interbeat intervals is used for developing the probabilistic modeling framework for the algorithm. An interbeat interval is the time elapsed between two successive R-wave peaks and is also known as an R-R interval or RRI.

A probabilistic model of a dynamical system observed through a point process can be used to meaningfully analyze heartbeat data. The heartbeat intervals are the times between R-wave events. These R-wave events correspond to the electrical impulses from the heart's conduction system, which initiate ventricular contractions. Therefore, the R-wave events form a point process because the events are a sequence of discrete occurrences in continuous time. Additionally, the autonomic nervous system is the principal dynamic system that modulates the dynamics of the heartbeat intervals. Thus, premature infant heartbeats can be accurately characterized by point process models of the R-R intervals.

The point process framework can be related to other variables, including respiratory activity, movement, pulse, and other related physiological variables. These relations may be used to establish new measures of control dynamics by the autonomic nervous system. A new statistical framework was developed using the indices obtained from the model. This combined framework combined measures sleep state, respiratory dynamics, and cardiovascular control for predicting life-threatening events in infants.

For any R-wave event uₖ, the waiting time until the next R-wave event obeys a history-dependent inverse-Gaussian probability density. This is expressed as f(t/Hᵤ_{ₖ},θ), where t is any time greater than uₖ, Hᵤ_{ₖ} is the history of R-R intervals up to uₖ, and θ is a vector of model parameters. The waiting time until the next R-wave event is also the length of the next R-R interval. The model is defined as:

$$f ( r \vert {H}_{{u}_{k}} , θ ) = \vert \frac{d t}{d r} \vert f ( t \vert {H}_{{u}_{k}} , θ ) = {[ \frac{{θ}_{p + 1}^{*}}{2 π r} ]}^{\frac{1}{2}} exp \{ - \frac{1}{2} \frac{{{θ}_{p + 1}^{*} [ 1 - {μ}^{*} ( {H}_{{u}_{k}} , θ ) r ]}^{2}}{{{μ}^{*} ( {H}_{{u}_{k}} , θ )}^{2} r} \} \qquad ( 9 )$$

where μ*(Hᵤ_{ₖ},θ)=c⁻¹μ(Hᵤ_{ₖ},θ) and θ*ₚ₊₁=c⁻¹θₚ₊₁. The mean and standard deviation of the heart rate probability density, respectively, are:

$$\begin{matrix}{μ}_{H R} = {{μ}^{*} ( {H}_{{u}_{k}} , θ )}^{- 1} + {θ}_{p + 1}^{{*}^{- 1}} & ( 10 ) \\ {σ}_{H R} = {[ \frac{2 {μ}^{*} ( {H}_{{u}_{k}} , θ ) + {θ}_{p + 1}^{*}}{{μ}^{*} ( {H}_{{u}_{k}} , θ ) \cdot {θ}_{p + 1}^{{*}^{2}}} ]}^{\frac{1}{2}} & ( 11 )\end{matrix}$$

The mean in Equation 9 becomes

$$μ ( {H}_{{u}_{k}} , θ , ρ , γ , η ) = {θ}_{0} + {\sum }_{j = 1}^{p} {θ}_{j} {w}_{k - j + 1} + {\sum }_{j = 1}^{q} {ρ}_{j} {RESP}_{k - j + 1} + {\sum }_{j = 1}^{m} {γ}_{j} SaO {2}_{k - j + 1} + {\sum }_{j = 1}^{s} {η}_{j} {MOV}_{k - j + 1} Λ > 0 \qquad ( 12 )$$

where RESP refers to the instantaneous lung volume measure, SaO2 refers to arterial-blood oxygen saturation, and MOV refers to movements monitored by electromyographic signals. The values of each are sampled in correspondence to the beat series because they are considered together with autoregressions on the R-R intervals. All other physiological signals act as covariates. Additionally, the amplitude of the respiration is included as one of the covariates because both the amplitude and the timing are important features to define the stability of breathing. It is contemplated that one or more of these covariates (e.g. the amplitude of the respiration) may be excluded from analysis.

Both the maximum local likelihood algorithm and the adaptive filtering algorithm were used to fit the model with covariates to the data. This allows for estimation of new indices of cardiovascular control defined as a function of the parameters θ=[θ₀ . . . θₚ], ρ=[ρ₁ . . . ρ_{q}], γ=[γ₁ . . . γ_{q}], η=[η₁ . . . ηₛ].

The model for interbreath interval is the same as discussed above with the mean interbreath interval defined by considering other physiological signals as covariates. The dynamics of poles of the auto-regression as well as the instantaneous power can serve as indices of the cardio-respiratory dynamics because the instantaneous mean is represented as an autoregressive process in both the interbreath interval model and the R-R interval model. The respiratory system was considered stable if the poles were inside the unit circle and unstable if the poles were outside of the unit circle. The degree of instability is defined using the number of poles outside the unit circle.

The resulting indices of cardio-respiratory dynamics are related to the life-threatening events including sleep state as a variable in the probability function. The model seeks to characterize the probability of onset of a life threatening event given the infant's physiological and autonomic state, as:

Pr(Apnea)=f(Sleep,Hₜ_{ₖ},θ,ρ,γ,η) (13)

This function was modeled using a framework including classifiers, regression analysis, principal component analysis, state vector machines, and adaptive filters, namely a Kalman filter. The function includes the indices defined for the R-R interval as well as interbreath interval. For the R-R interval model and interbreath interval models, a parametric approach was pursued. This approach characterized specific indices from the auto-regression models. The parameters were estimated using local likelihood and/or adaptive algorithms. The model fits were tested using well-established goodness-of-fit analysis. After determining the functions, indices extracted from this new explicit framework were used to statistically assess the predictive power of the model across the available database, both with and without vibrotactile stimulation.

The observations outlined above can be used to develop systems and devices that measure, indicate, and initiate other processes when a predetermined condition is met (e.g, a specified interbreath interval, R-R interval, and/or interbreath interval variability condition). The initiation of other processes can take many forms. One non-limiting example is to warn an individual when a predetermined condition is met or predetermined boundaries are crossed. The warning could include, for example, triggering an alarm, illuminating a light, initiating a sound, altering a display device such as a monitor, creating notes in medical records or chart recordings, sending a text alert such as an e-mail, SMS, or MMS message, and/or sending an automated phone call. Additionally, or alternatively, a corrective therapy can be automatically applied upon the happening of a predetermined condition. One nonlimiting example would be to initiate therapeutic vibration of a neonatal mattress for avoiding apnea or hypoxia. Moreover, a single device can perform multiple functions such as the example of a neonatal mattress with sensor, actuators, and computation incorporated measuring respiration of an infant and using algorithm and process described to initiate a therapy or action to stimulate and restore breathing.

The point process model was applied to an existing infant database in order to understand the respiratory dynamics related to mechanosensory stimulation. It was shown that the variance of the interbreath intervals is an important indicator of instability of breathing, with higher variance indicating irregular breathing and increased risk of apnea or hypoxia and lower variance indicating the stable breathing patterns and decreased risk of apnea or hypoxia.

It was expected that stimulation would induce rapid changes in interbreath interval variance because mechanoreceptor stimulation affects the respiratory oscillator via neural signals. Surprisingly, analysis of eleven infants revealed that the respiratory system exhibits relatively slow dynamics in interbreath interval variance in response to both initiation and removal of mechanoreceptor stimulation.

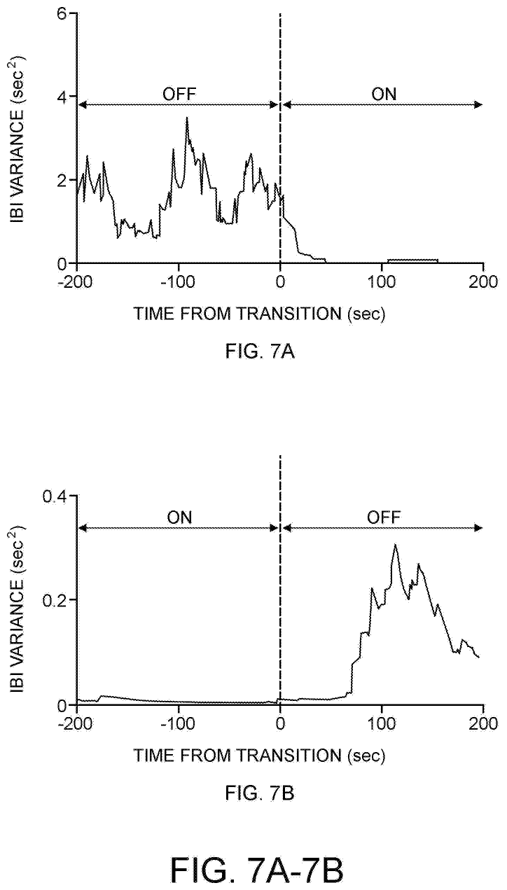

Referring now to FIGS. 7A and 7B, an example of the change in interbreath interval variance in response mattress stimulation is shown. FIG. 7A shows interbreath interval variance over time when the mattress stimulation was initiated. During times −200 to 0, no stimulation was present and the interbreath interval showed considerable variance. Stimulation was initiated at time 0. Once stimulation was initiated, the variance began to decline until no variance was noticed at approximately 60 seconds. Between 60 and 200 seconds there is almost no variance present.

FIG. 7B shows interbreath interval variance over time when the mattress stimulation was removed. During times −200 to 0, stimulation was present and the interbreath interval showed almost no variance. Stimulation was terminated at time 0. Once stimulation was terminated, the level of variance remained at almost zero until a sharp increase at approximately 60 seconds. Between 60 and 200 seconds, variance began fluctuating again. The study of eleven infants showed that the interbreath interval variance evolved to the new level within approximately one minute.

The interbreath interval data in FIGS. 7A and 7B was obtained by implementing the point process model of respiration. This revealed a parameter that is necessary for a device to prevent apnea. As shown in FIGS. 7A and 7B, impending apnea must be anticipated within approximately one minute in order to actuate the mechanosensory stimulus in time to prevent the apnea. Similarly, removal of the stimulus could result in persistent beneficial after-effects that maintain stability of breathing for up to approximately one minute after cessation of the stimulus. It is contemplated that this lag time might be different depending on factors such as post-conceptional ages, gestational age, concurrent conditions that might affect signaling within the respiratory control system, monitoring method, etc. The respiratory response time to stimulation onset and offset can be estimated for data sets from individual infants, and the resultant time constant can be automated and incorporated into the algorithm used to control the actuators that provide feedback mechanosensory stimulation to the respiratory control system.

In accordance with one embodiment, the present invention can be used to track the instability of breathing in infants, and in particular, preterm infants. Preterm infants with post-conceptional age of less than 36 weeks commonly have irregular breathing patterns with periodic and sporadic pauses in breathing. Variance has been shown to be a good marker for the incidence of apnea and hypoxia events.

It is essential to correctly quantify the irregularity of the breathing patterns, so that appropriate magnitude as well as duration of mechanosensory (vibrotactile) stimulation can be provided to improve the breathing patterns in preterm infants.

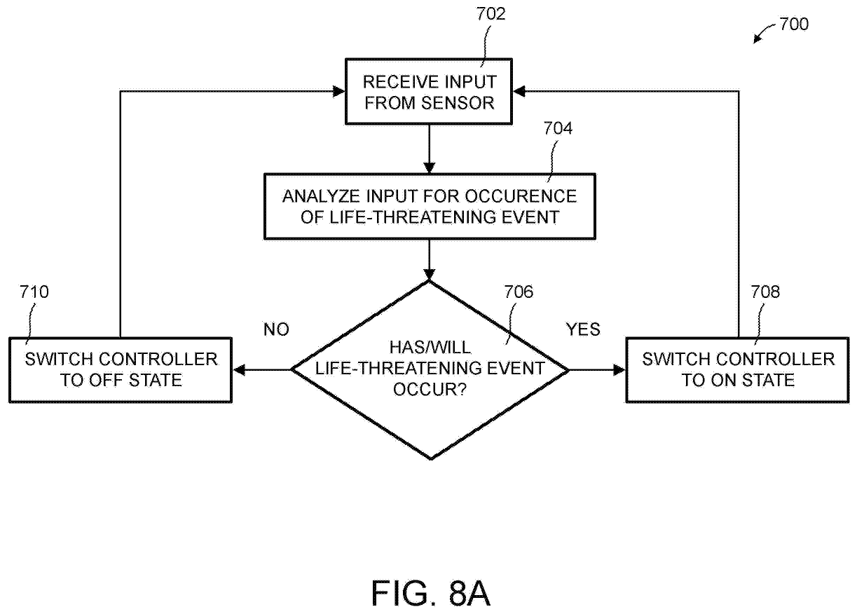

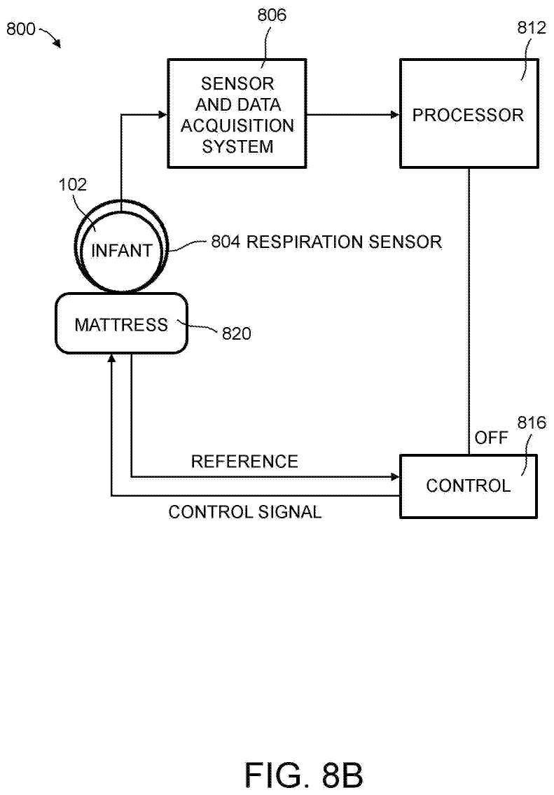

In accordance with one embodiment of the invention, FIG. 8A shows a flowchart for an algorithm 700 to monitor physiological instabilities in real time. Characterizing parameters (e.g. variance, heartbeat) can be used to assess likelihood of a life-threatening event occurring based on the monitored physiological factors. Step 702 receives input from sensors. By way of non-limiting example, these sensors can monitor heartbeat and/or breathing patterns. Step 704 analyzes the input for the occurrence of a life-threatening event. The occurrence of the life-threatening event may either be occurring contemporaneously with the analysis and monitoring, or it may occur in the future. By way of non-limiting example, a threshold value can be set while monitoring instantaneous breathing variance. At decision box 706, it is determined whether a life-threatening event has or will occur. By way of nonlimiting example, a threshold or set-point for variance indicates whether or not a life-threatening event has occurred. If the value is above a certain threshold, a life-threatening event has occurred.

If the algorithm detects that a life threatening event has or will occur, a controller is switched to the ON state at step 708. The controller is adapted to deliver vibrotactile stimulation to the source of monitored input (e.g. an infant). The algorithm 700 continues to receive input from the input sensor at step 702. It is contemplated that the controller may remain in the ON state for a predetermined amount of time, or until a precondition is met.

If the algorithm does not detect a life threatening event at decision box 706, the controller is biased to the OFF state at step 710. The algorithm 700 then continues to receive input from the sensor at step 702.

FIG. 8B shows a system 800 that monitors changes in breathing in real time according to one embodiment. The system 800 of FIG. 8B includes a respiration sensor 804, a sensor and data acquisition system 806, and a controller 816. The system 800 includes a vibrotactile stimulation mattress 820, which is connected to the controller 816. The respiration sensor 804 can be fastened to an infant 802 by, for example, a band or strap. The respiration sensor 804 measures the respiration of the infant 802. For instance, the respiration sensor may be a pulse oximeter measuring blood oxygenation or measure other aspects of the infant's respiratory function.

The sensor and data acquisition system 806 receives signals from the respiration sensor 804 and produces a respiration signal that is input to a respiration signal processor 812 of the controller 816. The respiration signal processor 812 uses the respiration signal to produce a variance value, a trend value, an average value (e.g. oxygen saturation). The value can be compared to a threshold or set-point by a compare module (e.g., software module, hardware component, comparator, etc.) and used to turn ON or OFF a mattress controller 816. The mattress controller 816 is generally biased in the OFF status, until the variance meets or exceeds the threshold. When the mattress controller 816 is in the ON state, the mattress 820 produces one or more stimuli to restore breathing. In some examples, the mattress controller 816 will turn on the mattress for an indicated amount of time (e.g. 30 minutes) in response to an average oxygenation saturation decreasing below a threshold or a trend indicating it will cross a threshold.

### Movement Features

A number of physiological perturbations result from spontaneous gross body movement, including increased oxygen consumption due to metabolic demands, movement induced hyperventilation and hypocapnea, and disruption of quiet sleep. These perturbations lead to a destabilizing effect on respiratory control and hence the occurrence of movement serves as an important physiological marker in predicting impending apneic and hypoxic events. What is more, gross body movements may be predictive of apneic events and hypoxic events for causal reasons such as movements that trigger hyperventilation, which in turn leads to hypocapnia. Gross body movements could also be predictive of apneic events and hypoxic events for symptomatic reasons such as movements that occur in response to a change in the underlying physiological state that is itself the causal factor leading to apnea.

Additionally, the quality of physiological data being monitored, particularly data obtained from respiratory and pulse plethysmogram signals, is adversely affected by gross body movements. These gross body movements are typically present in about one-quarter of recording times. Thus, features derived from gross body movement patterns can also incorporated into an analysis to improve prediction of apneic or hypoxic events.

Movement estimation is also useful in building statistical models of the joint feature distributions because it helps to explain the existence of movement artifacts in other measurement modalities. For example, one complication in interpreting previous prediction results on the same data set is the presence of movement artifacts in the IBI estimates. Surprisingly, the conflation of gross body movement with breathing signals indicates that the predictive value of respiratory measurements may be primarily due to information about breathing dynamics, primarily due to information about movement patterns, or even due to information about both.

One way to improve prediction of apneic and hypoxic events, or to improve prediction of overall or respiratory function (e.g. average blood oxygenation) is to incorporate gross body movement data into the analysis of physiological factors. Gross body movement data can be collected using independent sensors to detect movement, extracted from sensors measuring other physiological data such as IBI and/or RRI, or a combination thereof. In one nonlimiting example, sensors are used to directly collect gross body movement data. Preferably, sensors collect the gross body movement data without being in constant contact with the body of the patient.

In some aspects, the contactless sensor measures electrical signals in a conductive material placed proximate the patient. The electrical signals can include disturbances in an electrical field of the sensor that is caused by movement of the body of the patient. The conductive material could be included within a mattress, an array of individual sensors, a mat, a conductive plate, a probe, textiles worn by or covering the patient, etc. In one nonlimiting example, capacitive coupling between the patient's body and the conductive probe or surface is used to detect movement. In another nonlimiting example, a conductive plate or series of plates could be used to indicate motion due to a change in electrical capacitance between two specific plates.

In some aspects, the contactless sensor measures a change in force. That change in force can be detected using, for example, strain gauges or pressure sensors. The strain gauges and/or pressure sensors can be included within a mattress, an array of individual sensors, a mat, etc.

In some aspects, the contactless sensor includes an optical sensor. The optical sensor can be configured to detect electromagnetic radiation in the visible spectrum, infrared spectrum, ultraviolet spectrum, etc. In one nonlimiting example, the optical sensors detect changes in patterned light or a laser curtain.

In some aspects, the contactless sensor includes accelerometers, temperature sensing devices, gas sensing devices, and/or microwave-based Doppler sensors. Gas sensors can be used to detect the concentration of, for example, carbon dioxide. The level of carbon dioxide detected can then be correlated to increased or decreased levels of oxygen consumption.

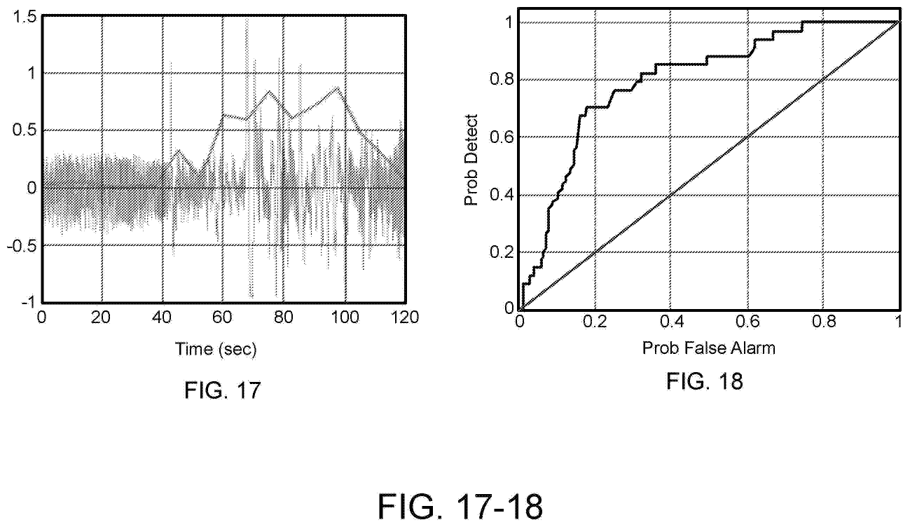

Additionally, or alternatively, gross body movement data can be derived from sensors configured to collect other physiological data. In one nonlimiting example, movement data is extracted from a pulse plethysmogram (PPG) signal. The PPG signal can be collected using, for example, a pulse oximeter attached to the patient. The output of the PPG includes both a pulse waveform and a power in low-frequency bands during movement of the patient. This output can be used to obtain a PPG-derived movement signal. The PPG-derived movement signal is obtained by calculating power of the low frequency band relative to total power of the PPG signal. The ratio is calculated to normalize the highly variable nature of PPG signals. FIG. 17 shows graph of an example pulse plethysmograph signal and a pulse-plethysmograph-derived gross body movement amplitude signal. As shown, the unit variance of the PPG signal varies rapidly between about 0.3 and −0.3 units from 0 seconds until about 40 seconds. Then, after about 40 seconds, the PPG signal begins to change its variance pattern and also increases the amplitude of variance, e.g., from about 1.5 to about −0.9 at about 70 seconds. The line placed over the PPG signal data is the PPG-derived movement amplitude signal. This signal is normalized and ranges between 0 and 1. As shown, the movement signal is approximately 0 until about 35 seconds, then rises to about 0.4 units at about 45 seconds. The movement signal continues to generally rise to about 0.8 units at about 75 seconds and about 95 seconds. The movement signal then begins to descend to about 0 units at about 120 seconds.

Statistical features can be used to describe the distribution of movement values. In some aspects, three statistical features are used. These features include the local mean μₘ and the standard deviation σₘ of the movement signal, as well as the ratio of these quantities. In some aspects, a denominator term is used to attenuate the ratio when the mean is small. The denominator term is shown in Equation 14.

$$\frac{{σ}_{m}}{0.001 + {μ}_{m}} \qquad ( 14 )$$

Six patients were tested using systems and methods of the present disclosure. The subjects were monitored for both respiratory and cardiovascular signals. The respiratory and cardiovascular signals were always used when both were available. A total of 2030 minutes of data was recorded across all six patients. Of that, only 15 minutes were discarded due to unavailability of either respiratory or cardiovascular sensor data. Additionally, physically implausible IBI and RRI values were automatically removed. The remaining values were then resampled at 10 Hz using shape-preserving piecewise cubic interpolation. The signals were then log-transformed and converted to standard units (zero mean, unit variance) for each patient. The log transformation made the IBI and RRI signals approximately normally distributed, and thus, well described by second order statistics.

A discrete plethysmograph signal was analyzed using a wavelet-based algorithm to derive information about gross body movement. The continuous wavelet transform of the discrete plethysmograph signal was determined as the convolution of the scaled and translated version of a mother wavelet. A Morlet wavelet was used to transform the data in time frequency plane. A Morlet wavelet is a plane wave modulated by Gaussian function. A dyadic representation of scales with eight sub-octaves per octave was used to obtain fine resolution in the time-frequency plane. The transformed data was used to calculate a wavelet power spectrum in a normalized scale. All peaks in the normalized spectrum were derived using a peak detection algorithm at each instant of time. These peaks were used to derive a dominant power time series. It was determined that the peak value in the range of about 0.8 seconds to about 5 seconds correlates with the strength and duration of the gross body movement seen in the data by using different time scales.

### Predictive Learning

Respiratory health, including average blood oxygenation, avoidance of life-threatening events—apnea, hypoxia, and bradycardia—can be improved by providing stimulation to the patient based on a trend or prediction about their future state. To achieve this goal, it is useful to anticipate these life-threatening events. Accurate prediction of future respiratory states (e.g. low blood oxygenation, apnea and/or hypoxia) can be achieved using a predictive learning paradigm that includes one or more of the following approaches: (A) Point process framework described in equation (1) as well as in equation (9) with other physiological data as co-variates as described in equation (12) along with predictor-corrector algorithm for predicting the state of the system within the point process modeling framework. (B) Standard learning models such as Gaussian Mixture Models (GMM), which are the combination of multiple Gaussian densities. (C) other predictive learning models such as statistical computing, pattern recognition, data modeling, data interpolation, data extrapolation and machine learning.

The above described predictive learning approaches employ historical data to improve the prediction performance. In the point process modeling this is achieved by introducing a history dependent function (the term H in equation (7)) whereas in the GMM models, historical data is used to train the model. In addition to the physiological data, the patient's sleep cycle and/or previous cycle can be used to define the history.

The inclusion of movement as a covariate to the point process modeling approach described in equation (12) results in improvement in the prediction of apnea. This framework provides a bivariate modeling framework with IBI or RR as one of the variables and movement as the other variables. In the bivariate framework, a linear relationship (coupling) between one variable with other variable can be derived. For example, in the case of IBI and movement, this framework will allow detection in the interaction between respiratory system and the system involved in gross body movement.

The indices defining these interactions are the instantaneous power, instantaneous coherence and instantaneous gain. Since the bivariate model is a parametric based approach, using the parameters of the model, indices can be obtained that provide the direction of interactions such as strength of coupling from IBI to movement as well as strength of coupling from movement to IBI, measured using instantaneous coherence and instantaneous gain.

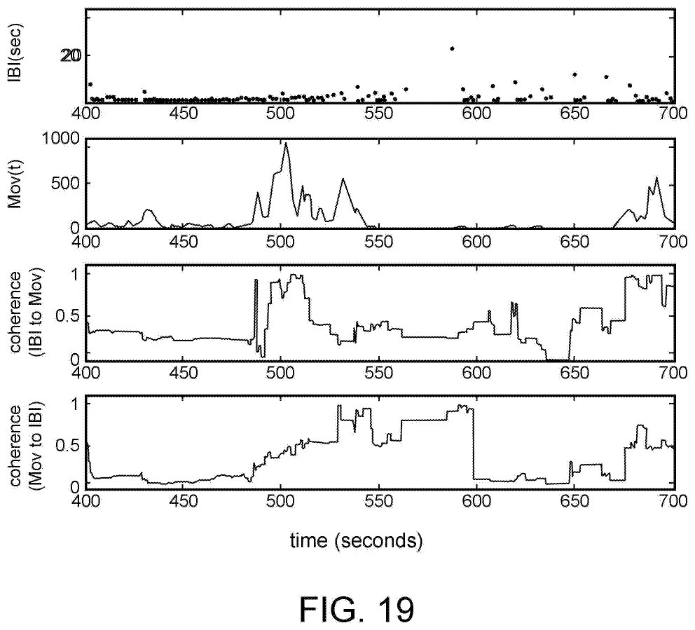

FIG. 19 demonstrates the relationship between IBI and the movement signal derived from the discrete plethysmograph signal. The first panel shows the IBI with an impending apnea around 590 seconds. However, prior to this apnea, there is a burst of movement signal shown in second panel between 480 to 550 seconds. Interestingly, this burst of movement can be considered as a predictor of apnea. The third and fourth panel shows the directional coupling of IBI to movement (third panel) and movement to IBI (fourth panel). Although there are interactions between IBI to movement during the movement burst, more predominant interactions are observed from movement to IBI and this interactions persists until the apnea event. Measurement in real-time of the directional coupling from the movement signal to the IBI signal as illustrated in FIG. 19 is an important predictor of apnea that can be used to prevent apnea before its occurrence.

The indices that quantify interaction between RR and movement can also be employed for the prediction of life threatening events, for example bradycardia events that accompany apnea and hypoxia or bradycardia events that occur in isolation. The framework in which point process modeling is embedded with bivariate modeling provides an important predictive learning framework for the prediction of life threatening events. Apnea is predicted by predicting parameters of the model using a predictive-corrector (Kalman filter type) approach by defining a state space and the output equation.

In the standard approach, physiological data is directly used to define a GMM model. Apnea as well as inter-apnea durations are used to train the model. The receiver operating characteristic (ROC) along with the area under the curve (AUC) is used as a metric for predicting the life threatening events for each patient.

### Machine Learning

Accurate prediction respiratory health events can be increased using machine learning analysis. Accurate prediction of impending events (e.g. low blood oxygenation) results in an increased efficacy of stimulation. In some aspects, prediction performance can be increased using a data set of a population. The data set can include physiological data from the population and can be analyzed using a machine learning analysis. In some aspects, prediction performance can be increased using a historical data set of the patient. The data set includes physiological data from the subject that was taken at earlier points in time. These earlier points in time can include the patient's present sleep cycle and/or previous sleep cycles.

One method to increase prediction performance is to adapt techniques from the field of automatic speaker recognition. The approach in automatic speaker recognition is to form a statistical background model from all speakers in a database, then to form a model tuned to a particular speaker using Bayesian adaptation from the background model. The statistical models can be GMMs that are weighted combinations of multiple Gaussian densities.

In some aspects, a separate background GMM is trained that is individualized to each patient. This GMM encodes the feature densities arising from all of the encountered physiological states of the patient. Bayesian adaptation can be used to form both a preapnea GMM and an interapnea GMM from this background model by using training data from the patient's preapnea and interapnea periods.

Evaluation of each adapted GMM model was performed using 40-fold cross-validation where the nearest training data to each test segment was separated by at least two minutes. The small data sets were made more robust using the combined likelihoods of an event from ten independently trained GMMs. Specifically, ten different background GMMs were obtained using independent random initializations. This resulted in the adaptation of ten preapnea and interapnea GMMs. The single-frame prediction score was then a 2-class log-likelihood ration obtained from the log of the sum of the ten preapnea GMM likelihoods minus the log of the sum of the ten interapnea GMM likelihoods. Multi-fram prediction scores were obtained by adding the preapnea and interapnea log-likelihood rations over time. This was done using the maximum cumulative sum statistic over a time interval of two minutes and fifty seconds.

The probability of prediction was analyzed by finding the fraction of eligible apneas that are predicted within the prediction time window when given a prediction threshold. The probability of false alarm is the number of interapnea frames that the prediction window is triggered divided by the total number of interapnea frames. Thus, increasing or decreasing the duration of the prediction window will increase or decrease both probabilities, respectively. The prediction time window was kept fixed at 5.5 minutes and a receiver operating characterizing (ROC) curve was obtained for each patient by varying the prediction threshold. The area under the ROC curve (AUC) was used as the evaluation metric. FIG. 18 illustrates a ROC curve generated from the prediction scores of all six patients used in the example study. The AUC in FIG. 18 is 0.80. This AUC was compared against AUC values obtained from random surrogates. The single-frame vectors were held constant while the apnea clusters were shuffled in time. One constraint placed on the shuffling was that all successive apneas that were separated by less than 9.5 minutes were assigned to the same apnea cluster. Another constraint was that the within-cluster inter-apnea time distances were kept constant. The between-cluster instances were randomly varied, but were kept greater than 9.5 minutes apart. 500 surrogate apnea profiles were generated for each patient. Machine learning, temporal integration, and prediction evaluation were done independently for each of the surrogate profiles. One-sided p values were computed by comparing the algorithm's AUC scores from the real data to the distribution of AUC scores obtained using the random surrogates.

Table 3 summarizes the apnea prediction results obtained from the six patients.

TABLE 3
APNEA PREDICTION PERFORMANCE

| Patient | RRI, Movement | RRI-IBI, Movement | Combined |
|---|---|---|---|
| No. | AUC (p) | AUC (p) | AUC (p) |
| 1 | .90 (.02) | .89 (.00) | 72.0 |
| 2 | .78 (.02) | .50 (.48) | 72.4 |
| 3 | .80 (.02) | .80 (.01) | 74.7 |
| 4 | .62 (.27) | .67 (.17) | 76.0 |
| 5 | .88 (.01) | .85 (.01) | 74.5 |
| 6 | .69 (.39) | .91 (.08) | 74.7 |
| 1-6 | .77 (.00) | .75 (.00) | 72.9 |

The left column of Table 3 summarizes the results when testing RRI features plus movement features. The middle column summarizes the results when testing joint RRI-IBI features plus movement features. The right column shows the results when the GMM likelihoods from these two feature combinations are summed prior to computing the single-frame log-likelihood ration. Combining both classifiers in this way produced the best results overall. Significance (p<0.05) was obtained on five out of six patients. The highest net AUC value was 0.80. These improvements are attributable to two factors. The first is the addition of a PPG-derived movement signal. The second is the replacement of a Gaussian classifier with a GMM classifier. These two factors may contribute approximately equally to the overall improvement.

### Isolation Mattress

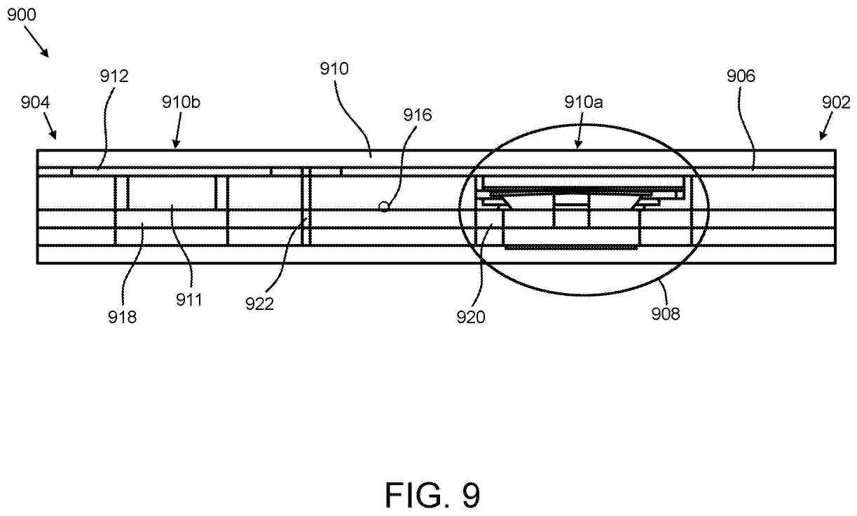

FIG. 9 depicts an isolation mattress 900 that applies isolated stochastic resonance mechanostimulation to a specific portion of the mattress according to one embodiment. The isolation mattress 900 includes a body 916. The body 916 includes an active region 902, a passive region 904, a top surface 910a, 910b, and a plurality of voids 918, 920, 922. The active region 902 includes an actuator 908 attached to an active soundboard 906. The passive region 904 includes an inertial device 914 attached to a passive soundboard 912. A passive-section void 918 is located around the inertial device 914. An active-section void 920 is located around the actuator 908. A soundboard void 922 is located between the active and passive soundboards 906, 912.

The active region 902 interacts with parts of an infant's body that can receive stimulation with little or no adverse consequences. These body parts include the legs and torso of the infant. The active region 902 is generally rectangular and occupies top surface 910a area, which is about two-thirds of the isolation mattress 900. It is contemplated that other shapes and sizes may be used be used to obtain the above described benefits.

The active soundboard 906 and the actuator 908 impart vibrational stimulation on the top surface 910a in the active region 902. The actuator 908 is attached to the active soundboard 906 such that movement of the actuator 908 moves the active soundboard 906. The active soundboard 906 is disposed below the top surface 910a such that at least a portion of the vibrations are imparted on the top surface 910a. For example, the active soundboard 906 can be placed approximately one-half inch below the top surface 910a. It is contemplated that other distances may be employed to achieve desired physical and vibrational properties of the top surface 910. For example, the soundboard may be placed from 0.4 inches to 0.6 inches, from 0.25 inches to 0.75 inches, from 0.1 inches to 1.0 inch, or even greater than 1.0 inch from the top surface 910.

The passive region 904 interacts with parts of an infant's body that are more sensitive to stimulation, such as the head. The passive region 904 is shown as being generally rectangular and occupies top surface 910a area, which is about one-third of the total top surface area of the isolation mattress 900. It is contemplated that other shapes and sizes may be used be used to obtain the above described benefits. It is additionally contemplated that the size of the active region 902 relative to the passive region 904 may be altered.

The passive region 904 is mechanically isolated from the active region 902. The inertial device 914 is attached to the passive soundboard 912 such that the inertial device 914 helps to dampen vibrations from the active soundboard 906 and actuator 908. In the illustrated embodiment, the inertial device 914 is a passive inertial device a mass attached to the passive soundboard 912. This mass is 660 g of aluminum rigidly attached to the passive soundboard 912. It is contemplated that the masses may be made of different materials or weights. It is also contemplated that the inertial device 914 may be a device that actively cancels vibrations imparted on the passive soundboard 912.

The body 916 may comprise various materials. By way of non-limiting example, an open-cell foam, gel, or other viscoelastic material may be used to damp the vibrations from the active soundboard 906 and the actuator 908. Additionally, the voids 918, 920, 922 assist in inhibiting vibrations from passing to the passive section. The passive-section void 918 prevents or inhibits vibrations from being imparted to the inertial device 914. The active-section void 920 prevents or inhibits the actuator 908 from imparting vibrations on the body 916. The soundboard void 922 prevents or inhibits vibrations from directly passing between the active soundboard 906 and the passive soundboard 912. It is also contemplated that any or all of the plurality of voids may be replaced with visco-elastic damping materials that alter and/or modify the transmission of vibrations from the active soundboard 906 and actuator 908 to the passive region 904. By way of non-limiting example, Young's Modulus, density, and/or visco-elastic properties may be considered when selecting materials. Sufficiently dissimilar material may result in improved isolation characteristics because vibration transmission between materials is a function of the area of contact in addition to the impedance of the materials to a specific type of vibration.

Additionally, the isolation mattress 900 may indicate the active and the passive regions 902, 904 to an individual. Examples of this include using visual indicia on the top surface 910, the body 916, and/or on a cover placed over the isolation mattress 900. The cover may be made from, for example, polymeric materials including medical grade vinyl.

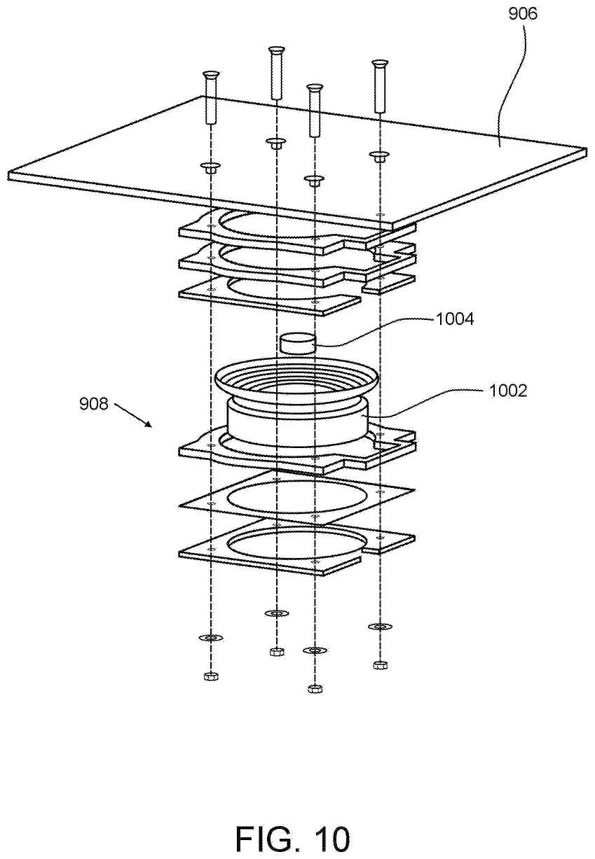

Referring now to FIG. 10, an exploded view of the actuator 908 is shown with the active soundboard 906 according to one embodiment. In the illustrated embodiment, the movement of the actuator 908 is obtained by imparting a drive signal to an audio driver 1002. A mass 1004 was added to the audio driver 1002 to increase output.

The isolation mattress 900 was tested against a single-bodied mattress. Both mattresses were 23 inches long, 12 inches wide, and 3.25 inches tall. All soundboards were located one-half inch below the top surface of the mattress.

The specifications for the single-bodied mattress included: an active soundboard being plywood; an actuator being a “woofer” audio driver of unknown origin; a body being a low-density foam rubber material; and the surface covering being a vinyl material.

The specifications for the isolation mattress 900 used in testing included: the active and passive soundboards 906, 912 being acrylic plastic; the inertial device 914 being a 660 g aluminum mass; the actuator 908 being an MCM model 1170 “woofer” audio driver that was modified to remove the driver cone and shorten the overall height; a 38.6 g mass 304 stainless steel mass was added to the audio driver; and the body was low-density polyurethane foam rubber material (UL94HF-1).

The first signal source consisted of a waveform generator connected to Class A/B current amplifier. This source was used to drive 2V peak-to-peak sinusoidal voltages in order to determine the transfer function of the isolation mattress 900 in the frequency band of interest. The frequencies used were: 10 Hz, 20 Hz, 30 Hz, 40 Hz, 50 Hz, 60 Hz, 70 Hz, 80 Hz, 90 Hz, 100 Hz and 200 Hz. These individual frequencies were used to de-convolve the system transfer function, but the results are not described herein. The second input source was a signal generator configured in the 30 Hz to 60 Hz range at various output settings (e.g. turns). Due to limited availability of the Balance Engineering generator for part of the testing, the third signal source consisted of ten 100 second recordings of the loaded output of the Balance Engineering generator from 1 turn to 10 turns (in 1 turn increments), sampled at 10 kSps, played back via National Instruments LabVIEW SignalExpress software and a National Instruments PCI-6281 Data Acquisition card connected a custom Class A/B current amplifier.

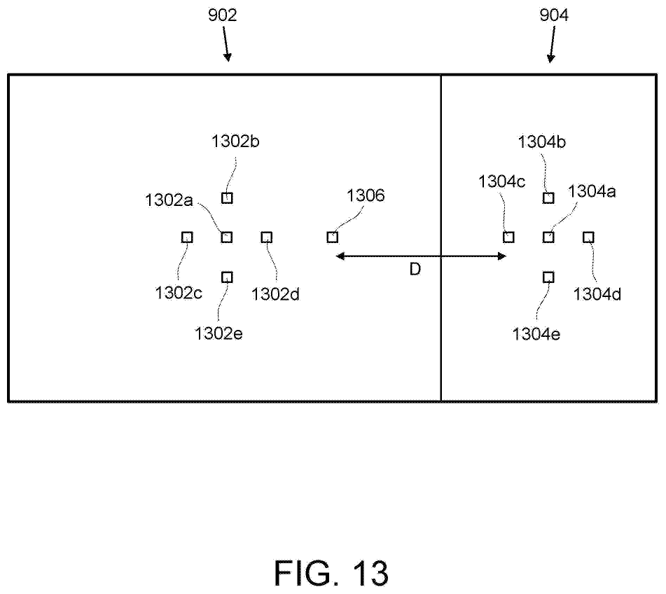

The isolation mattress was marked with reflective tape for accurate displacement measurements with the MTI-2100. As seen in FIG. 13, tape was placed at centers 1302a, 1304a of the active and passive regions 902, 904, respectively. Tape was also placed at points three inches above, to each side of, and below the centers 1302a, 1302b (1302b-e and 1304b-e, respectively) for a total of ten measurement locations. Measurements were also taken to determine the delivered stimulus and percentage isolation for the head if the infant were placed on the physical center point 1306 of the isolation mattress 900 rather than being placed on the center 1302a of the active region 902. Point 1304c was used to describe displacement at the infant's head because it was 5″ away from the mattress center 1306. As with the previous characterization, surface displacement measurements were collected using the MTI-2100 Fotonic Displacement system on an air table.

All measurements with the MTI-2100 system were taken using a Model 2062R fiber optic probe in its Range 1 measurement configuration. The linear range for the Model 2062R probe the Range 1 configuration was 152 μm with a nominal sensitivity of 0.025 μm. Each recording period was 100 seconds for every test, regardless of stimulus type. The output of the MTI-2100 system was recorded at 10 kSps and stored into a text file using a Tektronix MSO4034B digital oscilloscope. The stimulus drive voltage and drive current were also recorded at this frequency.

The recorded results were processed using MATLAB® in a similar manner to the methods of the previous characterization. Symmetric 3-pole high-pass Butterworth filters (cut-off of 1 Hz) and low-pass Butterworth filters (cut-off of 4 kHz) were applied to the data. The power spectral density was calculated using Welch's method with a spectral frame size of 1 Hz and a resolution sensitivity of 1.1 Hz. The Root-Mean-Squared value for output displacement was computed using a single window because it yielded more accurate results with less computational time than a sliding window of 0.1 seconds.

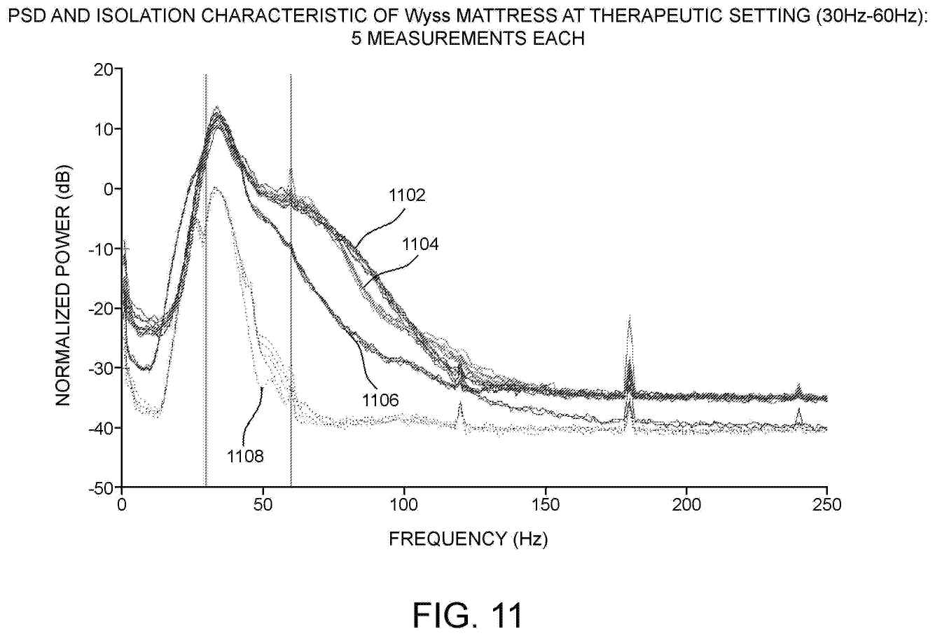

FIG. 11 shows results from the test of the single-bodied mattress compared to the isolation mattress with active and passive regions. The isolation mattress was the same as described in FIG. 9. Line 1102 represents readings from the tested single-bodied mattress at the center of stimulation for 1.5 turns. Line 1104 represents readings from the single-bodied mattress measured at the location of an infant's head for 1.5 turns. Line 1106 represents readings from the isolation mattress measured at the active region center 1302a at 2.75 turns of the signal generator, which was determined to produce the same therapeutic amplitude as the single-bodied mattress at 1.5 turns. Line 1108 represents readings from the isolation mattress measured at the passive region center 1304a at 2.75 turns. The output power spectral density of the isolation mattress closely matched the single-bodied mattress from 4 Hz-43 Hz, but the delivered power drops off from 44 Hz-60 Hz. The difference above 44 Hz may have been caused by the outer vinyl skin of the tested isolation mattress internally adhering to the body of the mattress. A similar attenuation was seen in previous single-bodied mattress characterization when a 1.5 kg mass was placed on the mattress.

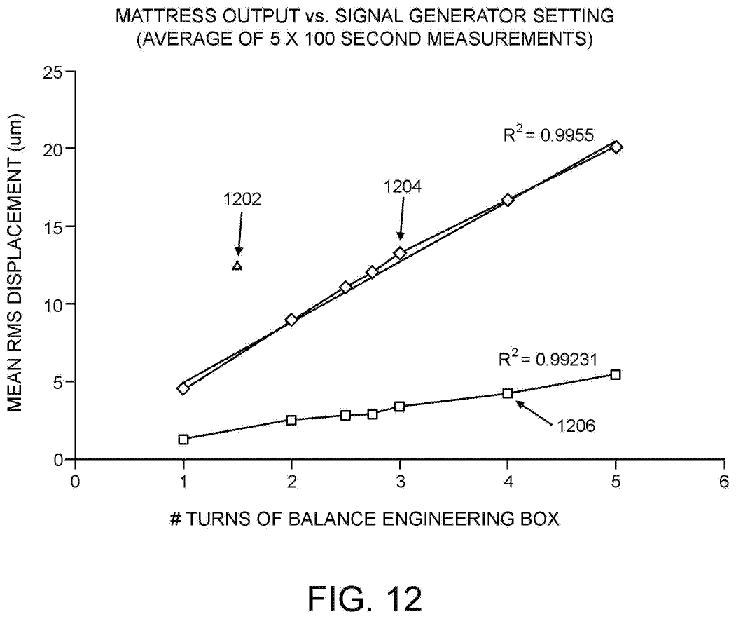

Referring now to FIG. 12, a graph of mattress output is shown. Point 1202 is the output of the single-bodied mattress. Line 1204 is the output of isolation mattress at the active region center 1302a. Line 1206 is the output of the isolation mattress at the passive region center 1304a. Table 1 lists the measured values shown in the graph with a calculation of the percent attenuation between the active region center 1302a and the passive region center 1304a.

TABLE 1
RMS Displacement Values and Percent
Attenuation for the Isolation Mattress

| | Mean Active | Mean Passive | Active |
|---|---|---|---|
| Stimulus | Region | Region | Center to |
| Generator | Center RMS | Center RMS | Passive Center |
| setting | Displacement | Displacement | Attenuation |
| [turns] | [μm] | [μm] | [%] |
| 1 | 4.5 | 1.3 | 72.0 |
| 2 | 8.9 | 2.5 | 72.4 |
| 2.5 | 11.0 | 2.8 | 74.7 |
| 2.75 | 12.1 | 2.9 | 76.0 |
| 3 | 13.2 | 3.4 | 74.5 |
| 4 | 16.7 | 4.2 | 74.7 |
| 5 | 20.1 | 5.5 | 72.9 |

As shown in table 1, there was a drastic reduction in displacement between the active center and the passive center. The attenuation between the centers was consistently between 72% and 76% across the tested range. That is, the isolation mattress 900 prevented approximately three quarters of the stimulation of the active region from reaching the passive region.

The secondary positions 1304c, 1306 provide data related to the attenuation of vibration between the approximate the head and body positions of an infant placed on the isolation mattress. Table 2 compares attenuation between an infant's head and body using the above described single-bodied mattress and the isolation mattress 900.

TABLE 2
Comparison of Single-bodied and Isolation Mattresses

| | Stimulus | Mean Mattress | Mean | |
|---|---|---|---|---|
| | Generator | Center RMS | Head RMS | |
| | Setting | Displacement | Displacement | Attenuation |
| | [turns] | [μm] | [μm] | [%] |
| Single- | 1.5 | 12.5 | 11.0 | 12.2 |
| bodied | | | | |
| Isolation | 2.75 | 8.4 | 2.6 | 69.5 |

Comparing the attenuation of the overall mattress center to the approximate head location for both mattresses resulted in the isolation mattress showing an improvement of 5.7 times over the single-bodied mattress.

The therapeutic level of stimulation of the single-bodied mattress was determined to be 1.5 turns of the amplifier on the noise generator as determined by comparison to previous tests. Therapeutic level of stimulation may be any stimulation that is capable of altering a sleep state or physiological function of sufficient amplitude to cause harm or or pain. This includes subthreshold, subarousal, and/or suprathreshold stimulation. The isolation mattress was tested to determine the turns needed to achieve an equivalent level of output stimulation. It was determined that 2.75 turns were the appropriate therapeutic setting for the isolation mattress. At this setting, the mean root-mean-squared displacement of the center 1302a of the active region 902 is comparable to the therapeutic displacement of the geometric center of the single-bodied mattress.

Sensors for direct monitoring and/or control of mattress surface displacement may be incorporated with the isolation mattress 900. These sensors can include, for example, embedded accelerometers or other vibratory sensors (e.g. pressure sensors, load cells, optical sensors). Such sensors can be used, for example, in modifying the drive signal for the active region in response to weight, loading, or the location of the infant on the mattress. Such sensors can be used, for example, in alerting caregivers to malfunctions or even active cancellation of stimulation in the passive region.

### Focal Stimulation

In another embodiment, focal stimulation may be used to apply stochastic resonance stimulation to a subject. Systemic vibration may be potentially inappropriate for patients who are at risk of intra-ventricular hemorrhage. Instead, focal stimulation can be used to both discover and target the correct mechanoreceptors to therapeutically address different modes of respiratory instability. Additionally, focal stimulation can deliver only the essential stimulation when required. Focal stimulators may be used to apply mechanical stochastic resonance stimulation to improve the respiratory function of infants at risk of apnea or other respiratory instabilities. The stimulation may be applied in both open- and closed-loop fashions.

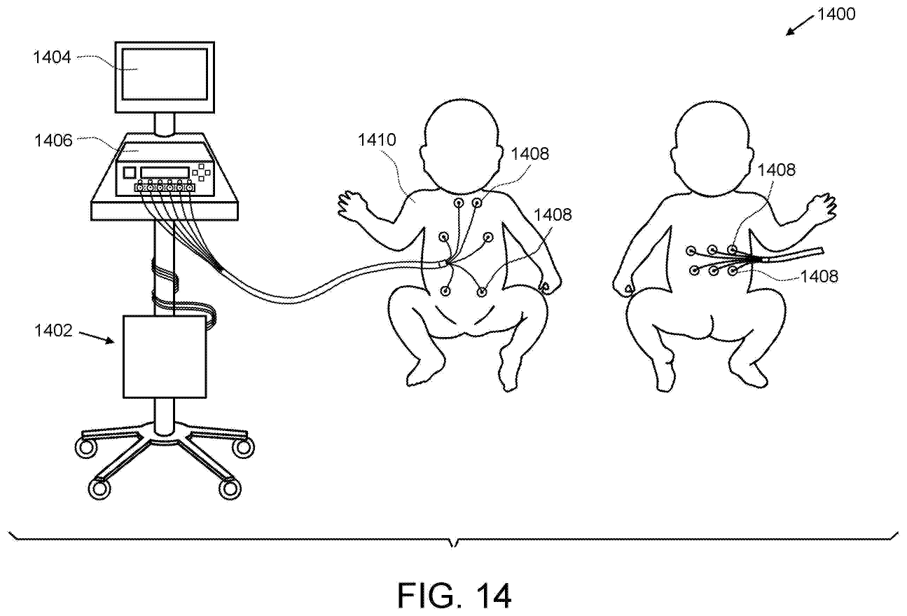

Referring now to FIG. 14, a focal system 1400 is shown according to one embodiment. The system 1400 includes a processor 1402, a user interface 1404, a signal generator 1406 and a plurality of focal stimulators 1408. The focal stimulators 1408 are applied to a body of a subject 1410 to stimulate to the subject. The system may additionally include a communications bus, data logging mechanism, and/or connections for input sensors. The communications bus provides an interface to attach external master controllers such as a laptop to the system 1400. The data logging mechanism may be used to locally store and/or report data. Input sensors such as temperature sensors, accelerometers, strain gages, pulse-oximeters, plethysmographs and other physiologic monitoring sensor systems may interface with the system to provide physiological information related to subject. This physiological information may be monitored and used by the system to initiate or alter stimulation.

The focal stimulators 1408 may be comprised of one type or a combination of types of actuators including electromagnetic, electromechanical, solid state actuators (e.g., Nitinol, piezoelectric), hydraulic, pneumatic, ferrofluid, electroactive polymer, etc. In the illustrated embodiment, the plurality of focal stimulators 1408 is designed to be placed in direct contact with the subject's skin. Thus, in this embodiment, it is desirable for the focal stimulators 1408 to be formed from biocompatible and/or hypoallergenic materials. For safety, the focal stimulators may also include double-electrical insulation so that the subject is protected from electrical discharge or electromagnetic interference.

The signal generator 1406 drives the focal stimulators 1408 and may drive them individually, in groups, or even as one unit. The signal generator 1406 may be, for example, a stochastic resonance noise generator and may include adjustable drive capabilities to ensure the delivery of adequate stimulation. The needed signal may be affected by conditions such as the stimulators being placed in an intervening brace or other mediating material. The focal stimulators 1408 may be applied to the subject using a number of materials such as braces, fitted garments, elastic bands, FDA-approved adhesives, etc.

The system 1400 may be used to control and optimize focal stimulation in response to an infant's real-time physiological status. For example, the system may monitor the infant's respiratory pattern and initiate stimulation to prevent or inhibit the occurrence of an impending apneic event or an impending hypoxic event. Additionally, the system 1400 may be used in developing algorithms to control and optimize focal stimulation. The use of physiological input sensors allows the device both to self-calibrate and deliver the correct stimulation independently of the attachment method and to dynamically adapt that stimulation during use.

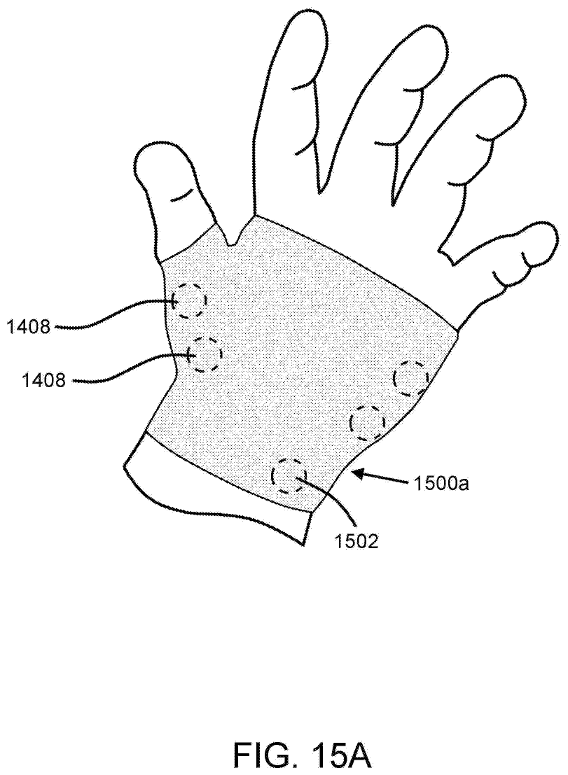

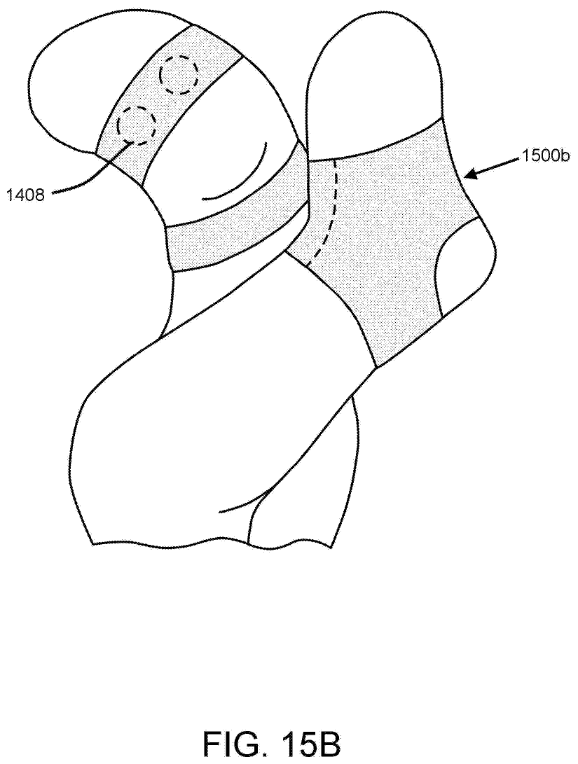

Referring now to FIGS. 15A and 15B, non-limiting examples of support garment structures for embedded focal stimulators are shown. Support garment structures may be made of a variety of materials including flexible materials such as neoprene, latex, rubber, silicone, cloth, wool, vinyl, polyvinyl chloride, nitrile, neoprene, knit textiles, composites, or leather. FIG. 15A shows a hand support structure 1500a that fits on the hand of an infant. The hand support structure 1500a includes a plurality of focal stimulators 1408 configured to apply stimulation to an isolated body part of the infant. In the illustrated embodiment, the body part is the infant's hand. Additionally, the hand support structure 1500a includes an input sensor such as, temperature sensors, blood pressure sensors 1502, accelerometers, strain gauges, pulse-oximeters, plethysmographs, and other physiological monitoring sensor systems that will assist in enabling the embedded focal stimulators 1408 during an apneic event or hypoxic event. FIG. 15B shows a foot support structure 1500b that fits on the foot of an infant and includes embedded focal stimulators 1408.

It is contemplated that the system may be condensed to a single embedded controller. The embedded controller includes algorithms developed to optimize the stimulation level and stimulation timing, and includes the integration of multiple types of sensors. The embedded controller may autonomously control the application of stochastic resonance stimulation based on either input sensors or a physician's programmed therapeutic regimen. These input sensors monitor at least one physiological condition. The placement and method of attachment of the focal stimulators 1408 also factor into the algorithm for the application of stimulation. Such a system may be condensed, simplified, and battery powered so that it may be designed for safe and efficacious use in home environments. Additionally, portions of the system such as sensors may communicate wirelessly with other portions of the system to decrease wires and increase safety.

### Array Stimulation

In yet another embodiment, array stimulation may be used to apply stochastic resonance stimulation to a subject. Array stimulation can be used to deliver targeted stimulation while covering an area for potential stimulation. Additionally, array stimulation can deliver synchronized stimulation patterns over the array. Array stimulators may be used, for example, to apply stochastic resonance stimulation to improve the respiratory function of infants at risk of apnea or other respiratory instabilities. The stimulation may be applied in various ways such as single-actuator stimulation, multiple-actuator stimulation, or even coordinated stimulation such as stroking.

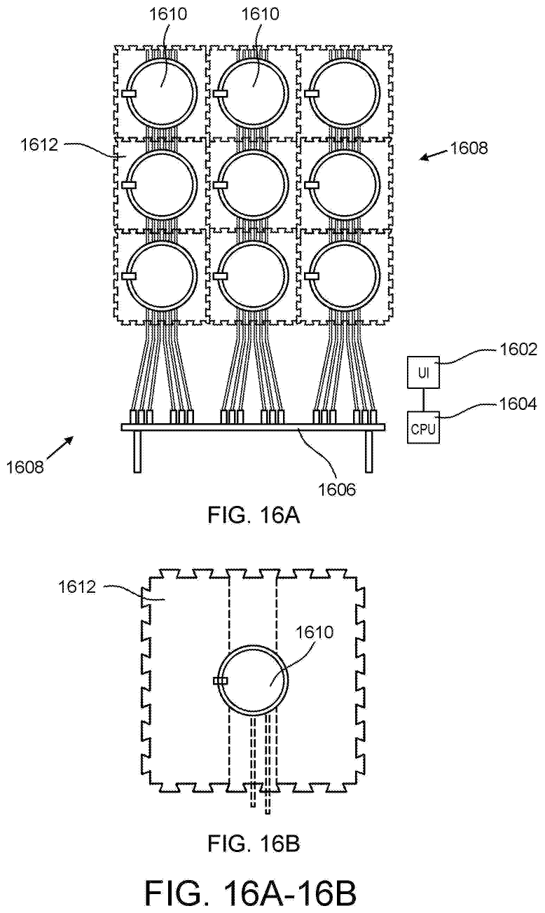

FIG. 16A depicts a stimulation array system 1600 according to one embodiment. The stimulation array system 1600 includes a user interface 1602, a processor 1604, a controller 1606, and a stimulation array 1608. The stimulation array includes stimulators 1610 to stimulate a subject. Other components may include a communications bus, data logging mechanism, and/or connections for input sensors.

The user interface 1602 allows the user to interact with the stimulation array system 1600 and is operatively connected to the processor 1604. The processor 1604 is operatively connected to the controller 1606. The controller 1606 is operatively connected to the stimulation array 1608 and drives the stimulators 1610. In this embodiment the stimulators 1610 are driven independently. It is contemplated that the stimulators 1610 may also be driven in groups.

In this embodiment stimulation array 1608 includes interlocking pieces 1612. Each interlocking piece 1612 includes a single stimulator 1610. By way of non-limiting example the stimulators may be electromagnetic, electromechanical, solid state actuators (e.g., Nitinol, piezoelectric), hydraulic, pneumatic, ferrofluid, electroactive polymer, etc. It is contemplated that more than one stimulator 1610 may be included on an interlocking piece 1612. It is additionally contemplated that the stimulation array 1608 may be a single mat.

The array system 1600 may be used to control and optimize focal stimulation in response to an infant's real-time physiological status. For example, the system may monitor the infant's respiratory pattern and initiate stimulation to prevent or inhibit the occurrence of an impending apneic event or an impending hypoxic event. The use of physiological input sensors allows the device both to self-calibrate and deliver the correct stimulation independently of the attachment method and to dynamically adapt that stimulation during use.

Additionally, the array system 1600 may include sensors to detect the location of a child on the stimulation array 1608. Detecting the location of the child allows the array system 1600 to target stimulation. This targeted stimulation can be used to deliver stimulation only to portions of the stimulation array 1608 occupied by the child, simulate a stroking motion, or simulate a wave motion. Additionally, detecting the location may also be used to determine orientation of a child. Determining orientation would allow for targeted stimulation of the child's body without stimulating the child's head regardless of the child's location. The sensors to determine location may be included with the stimulation array 1608 or may be independent of the stimulation array 1608.

In accordance with the above embodiments, the vibrotactile stimulation can be turned on and turned off for a predefined periods of time. Alternatively, the vibrotactile stimulation can remain on until a change in one or more aspects of the breathing pattern are detected. Further, the nature of the stimulation can change over time such that the amplitude, frequency characteristics, and/or period of vibration can change over time.

### Stochastic Stimulation to Ventilated Infants: Study 1

The inventors additionally performed a clinical study that tested the hypothesis that stochastic stimulation could provide additional benefits beyond the encouragement of breathing pacemaker neuron drive. During the study, ventilator dependent infants were placed on the mattress with 30 minute on/off cycles. Ventilator dependent infants are an interesting population because they can suffer hypoxia and oxygen instability episodes even though their breathing rates are stabilized by a machine. In a preliminary analysis, the stimulation decreased the duration of the hypoxia by 30% (p=0.04) and decreased the variance in oxygenation (SaO2) by 20% (p=0.025) when compared to the non-stimulation period. This is a novel finding since the previous effect was thought to be purely caused by encouraging the pacemaker drive.

Recruitment of subjects was guided by strict criteria that included preterm infants with a gestational age of <36 wks and undergoing conventional mechanical ventilation treatment for respiratory distress for at least 24 hours. Indication for initial treatment by intubation included respiratory distress syndrome (RDS), apnea of prematurity leading to severe or intractable apnea, hypercapnia, or significant respiratory compromise requiring respiratory support. Exclusion criteria were as follows: evidence of severe pulmonary disease requiring steroid courses at time of study (e.g. bronchopulmonary dysplasia), acidosis or cord pH<7, hydrocephalus or intraventricular hemorrhage (IVH)>grade 2, congenital abnormality affecting respiration, anatomic brain anomaly, seizure disorder, clinically significant cardiac shunt, anemia (hemoglobin<8 g/dL), pneumothorax or lung injury, and ventilation related to post-operative care. Infants treated with caffeine or other xanthines were included if the drug had reached a steady state level.

Eleven infants participated in twelve studies at the University of Massachusetts Memorial Newborn Intensive Care Unit. All infants studied were receiving support via the Drager Evita XL Ventilators used in the UMass Memorial NICU (Dragerwerk AG & Co, Lubeck, Germany). Signals were collected from the ventilator, the infant's bedside monitor (Philips Intellivue MP70, Philips Medical Systems, Eindhoven, Netherlands) and bedside capnograph (Cosmo Plus System, Novametrix Medical Systems, Wallingford, Conn.). Signals from the ventilator and capnograph were recorded through the patient monitor using VueLink modules (Ventilator Module and AuxPlus B Module, Philips Medical Systems, Eindhoven, Netherlands). Acquired waveforms from the patient monitor included EKG, respiration, plethysmography and arterial blood pressure (ABP). Airway pressure (AWP) and airway flow (AWF) waveforms were taken from the ventilator, and CO2 and airway volume (AWV) waveforms were taken from the capnograph. Monitor numeric signals acquired included percent blood oxygenation (SpO2), respiration rate (RR), heart rate (HR), pulse rate, perfusion as well as systolic, diastolic and mean values of arterial blood pressure when available. Numeric signals from the ventilator included mean airway pressure, fraction of inspired oxygen (FiO2), inspiratory time, positive end expiratory pressure (PEEP), spontaneous respiration rate (spRR) and ratio of inspiration time to expiration time. Capnograph numerics included end-tidal (et) CO2, volume of CO2 exhaled per minute, mixed expired CO2, expired tidal volume, peak inspiratory pressure (PIP) and total minute volume (tMV).

All acquired waveforms were sampled at a rate of 125 Hz, whereas all numeric signals were sampled at 1 Hz due to the limitations of the patient monitor. Data was recorded and displayed in real time using a personal laptop with data acquisition software (Trendface, Ixellence GmbH, Wildau, Germany). Data was de-identified and transferred to a secure server.

A specially designed mattress was placed underneath the infant for the duration of the study. The original construction and mechanics of an earlier version of this mattress was fully detailed in a previous manuscript (Bloch-Salisbury et al. 2009). For this study, a newer design featuring attenuated stimulation was applied within the upper one third of the mattress or ‘isolation zone’ to minimize cranial vibrations. Construction design utilized a low frequency woofer voice coil rigidly attached to a sounding board. Mechanical separation of the ‘stimulus zone’ from the ‘isolation zone’ was accomplished by selecting firm viscoelastic open cell foam for the bulk, splitting the sounding board into two regions, adding mass to the isolation zone sounding board to provide passive inertial damping and creating voids in the bulk of the mattress to prevent lateral transmission of vibration. The output performance of the new dual-zone mattress was characterized by driving the system with a Balance Engineering stimulus generator and recording the resulting surface displacement of the mattress using an MTI-2100 optical measuring system. Measurements were taken at 10 locations: five locations in each zone, including the geometric centers of the stimulus and isolation zones. A 72% torso-to-head attenuation factor was found between the two zones, or a 5.7× improvement from the original mattress design.

Further measurements confirmed the ‘stimulation zone’ continued to deliver the previously described therapeutic stimulus: 30-60 Hz and 12 microns RMS+/−10%. Furthermore, this mattress has an identical size, covering and apparent firmness to the original mattress.

Study setup was initiated between 7 AM and 8 AM which was dependent on the infant's feeding schedule. During nursing assessments, the mattress was placed underneath the infant and all equipment setup was completed. Care was taken to ensure the infant was positioned correctly below the mattress head isolation zone in that stimulation was only administered below the shoulders. The infants were then fed and were allowed to rest for 30 minutes prior to starting the protocol. Once the post feeding stage had elapsed, the mattress was either left off or turned on providing gentle stimulation to the thorax (this was randomized between infants). Alternating 30 minute epochs of stimulation or no stimulation continued for two hours at which point the morning session was ended in preparation for the midday feeding and assessment.

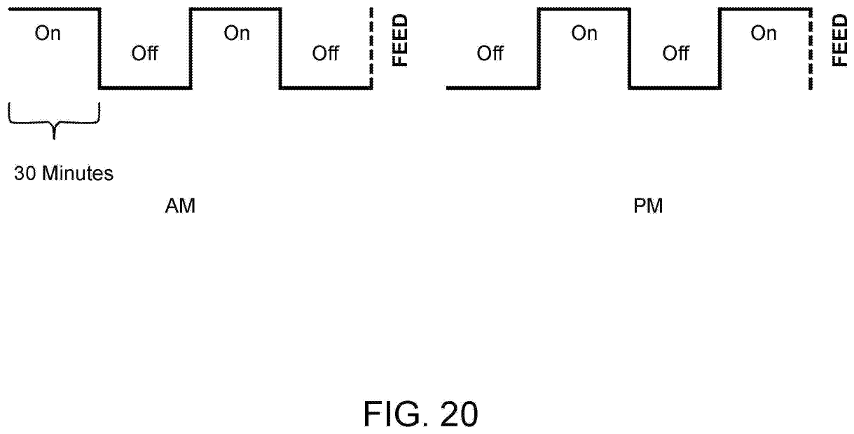

FIG. 20 illustrates a graph showing the condition protocol for each infant throughout the study. This protocol was repeated after a second post feeding stage, and the equipment was removed during the next assessment period in an effort to disturb the infant as little as possible. One infant was removed for Skin to Skin Care during the midday gavage feeding.

Data recorded in TrendFace was exported to separate program for full analysis (LabChart 7 Pro, AdInstruments, Colorado Springs, Colo.). For each subject, the files were parsed into on and off sessions and analyzed as 30 minute bins. Mean FiO₂. etCO₂, SpO₂, HR, peak AWP and mean AWP were calculated using the software's data pad feature. Furthermore, the infant's spontaneous breathing was found by subtracting the frequency of administered mechanical breaths by the frequency of all recorded peaks on the ventilator AWP signal. Standard deviation was also calculated for each signal using the statistics function found in the software.

O₂ desaturation periods were noted as instances where the SpO₂ measurement fell below an 85% threshold. The frequency and duration of these events were manually found and recorded. O₂ data has been presented as a percentage of valid recording time (i.e. with interventions excluded). Because the infrared transducer clinically used to record blood oxygen levels has a well-documented delay, the first 10 seconds of the SpO₂ signal was removed from analysis at the beginning of each epoch regardless of condition.

Criteria for exclusion of brief data periods is as follows: any handling of the infant affecting signal recording or generating extreme movement (i.e. repositioning, diaper changing, endotracheal suctioning, extended blood gas testing, or brief loss of signal). Data across all channels during such events were not included in analysis and was thus deducted from the calculated valid recording time for the appropriate 30 minute period. Special circumstances for SpO₂ data exclusion occurred when desaturation episodes spanned changing conditions (for example, the desaturation began in an ON condition and resolved during the OFF condition). In these cases, the desaturation was totaled during period within which it started and the portion continuing into the next condition was excluded from all totals.

For analysis of data variables with two components, paired t-tests were used to determine statistical difference. This was used to report differences in parameters between ON and OFF conditions. The Pearson coefficient was also used to determine correlation and directionality between stability of oxygenation and infant weight. Values are expressed as means and standard deviation. Values of P<0.05 were considered to be statistically significant. Graphical summaries illustrating results of all subjects utilized a plotted ratio of mean values in the ON and OFF conditions. Percent reduction of the variable is reported as 1 minus the ratio (×100). A 95% confidence interval is also reported to confirm the range of probably mean values.

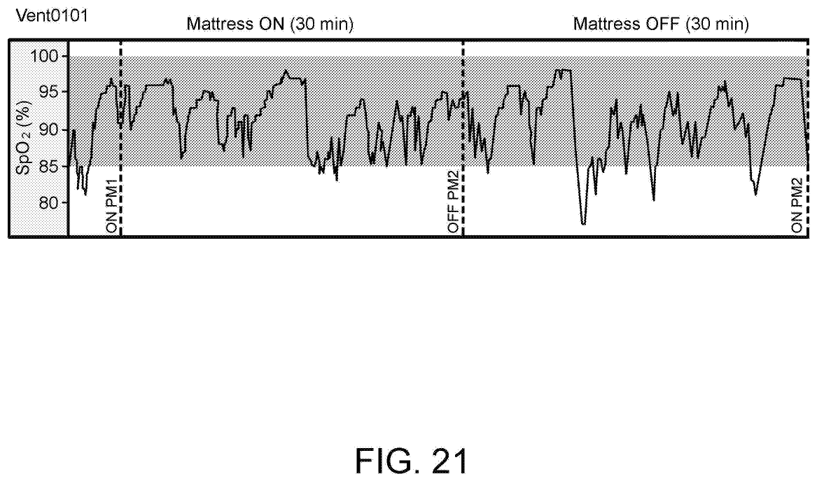

FIG. 21 is an example of improvement in a single infant over one hour where the condition has been changed from stimulus ON to stimulus OFF. The threshold band indicates a range of 100%-85% oxygen below which was considered a desaturation event. This shows the increased variability of the infant's O₂ saturation as the mattress is switched into the OFF condition, indicating worsening oxygenation.

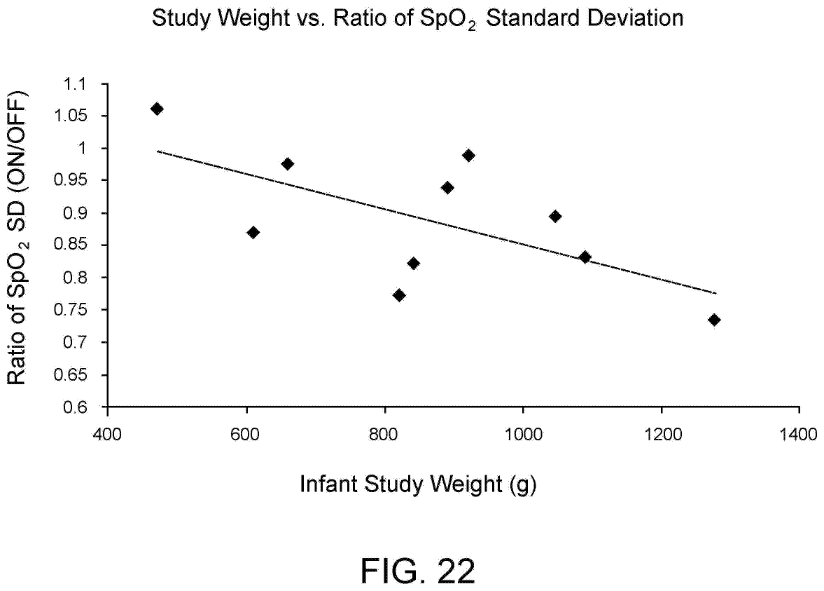

Study weight was shown to be related to effect of stimulation as calculated by the ratio of SpO₂ standard deviation for the on versus off mattress periods for the same infant. Specifically, as illustrated in FIG. 22, the effect of therapeutic stimulation may be reduced for very low birth weight infants prompting the consideration of adjustments in stimulation based on infant mass.

### Respiratory Support Apparatus

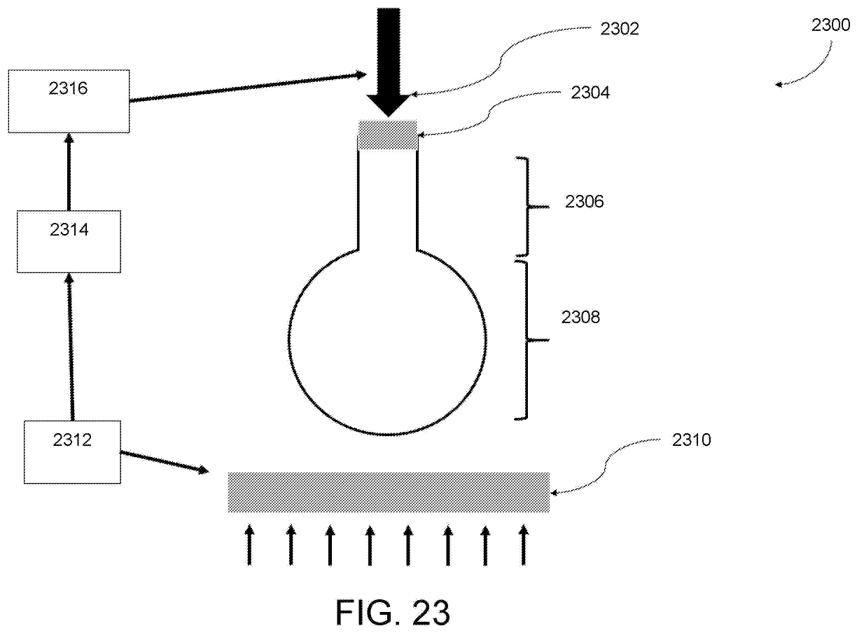

The present disclosure can also provide for an additional system to provide artificial respiratory support for infants. The system can use the stochastic signal which drives the mattress actuator (according to FIGS. 9 and 10) to optimize alveolar gas exchange inside respiratory support device. FIG. 23 shows an exemplary respiratory support system 2300, according to an embodiment of the present disclosure. The respiratory support system 2300 can include a pressure support system 2302; an infant facial attachment 2304; an airway passage 2306; a gas exchange compartment 2308; a stimulation mattress 2310; a stochastic signal 2312; a controller 2314; and an altered signal 2316.

A stochastic signal 2312 F(t) can control a vibration of the isolation mattress 2310, in accordance with various embodiments of the present disclosure (including, for example, the descriptions of FIGS. 8A-10). The stochastic signal 2312 F(t) can be used by a mattress controller to determine when vibrotactile stimulation of a mattress 2310 should be used to restore an infants breathing based on received input from sensors on an infant (not pictured).

The stochastic signal 2312 can be split to provide a duplicate signal as input to a controller 2314. The controller 2314 can provide adjustable parameters derived from the stochastic signal 2312 to produce (1) a time-shift of the stochastic signal 2314 by a specified amount τ, (2) an offset value δ, and (3) a low- and high-pass filter. These adjustable parameters contribute to an altered signal 2316 F′(t+τ)+δ which the controller 2314 can send to drive the pressure support mechanism 2302.

In order to determine the altered signal 2316 F′(t+τ)+δ, numerical modeling can be used to approximate the alveolar gas exchange. For example, the altered signal 2316 can be approximated through a geometric representation of the alveoli or a fluid structure interaction. Numerical modeling can be clinically suboptimal because the modelling is not personalized to the infant receiving treatment and does not account for biomechanical parameters such as age, gender, and specific disease states. Bronchopulmonary dysplasia, in particular, causes reduced compliance and increased airway resistance which can complicate how an infant receives and responds to the positive pressure support mechanism 2302. Therefore, in some embodiments of the present disclosure, model-based estimations can include clinical measurements to provide a first approximation for the altered signal 2316 F′(t+τ)+δ.

In another embodiment of the present disclosure, model-free optimization methods can be used to determine the altered signal 2316 F′(t+τ)+δ. An extrema distortion method can estimate the altered signal 2316 F′(t+τ)+δ by optimizing a measured outcome. For example, the measured oxygen saturation can be optimized using standard pulse plethysmography. Clinicians typically aim for an optimal range of oxygen saturation and so an extrema distortion method can iteratively shape an input stimulus to achieve the desired outcome. For example, an optimal range of oxygen saturation can be 88-94% for infants with a post-conception age of 30 weeks. The stochastic signal 2312 F(t) can be iteratively reshaped towards the altered signal 2316 F′(t+τ)+δ to achieved the desired range of oxygen saturation. The reshaping can also account for minimizing an energy transfer of, such as minimizing a root-mean-square of air pressure fluctuations of the pressure support system 2302 with a fixed offset δ varied between 5 and 10 cm H₂O.

Referring back to the respiratory support system 2300 of FIG. 23, the pressure support mechanism 2302 can move breathable air into an infant's lungs by adapting pressure and flow characteristics of air based on the altered signal 2316. The air pressure can be configured to enter an infant facial attachment 2304. The infant can be configured to breathe the provided air pressure through the infant facial attachment 2304. The infant facial attachment 2304 can also be connected to an airway 2306 and an alveolar gas exchanging compartment 2308. The alveolar gas exchange can be optimized in the compartment 2308 according to the altered signal 2316 and a clinical index such as the alveolar-arterial oxygen gradient.

Therefore, the exemplary respiratory support system 2300 can adjust the alveolar gas exchange via a stochastic signal to provide appropriate air pressure to an infant. The respiratory support system 2300 can coordinate air flow with stochastic mechano-perturbations delivered through a mattress. For example, the air pressure can be increased at the same time the patient receives a mechanical stimulation. In some examples, there might be a cyclical air pressure increase and decrease with a maxima of the cycle coinciding with a mechanical stimulation.

### Computer and Hardware Implementation of Disclosure

It should initially be understood that the disclosure herein may be implemented with any type of hardware and/or software, and may be a pre-programmed general purpose computing device. For example, the system may be implemented using a server, a personal computer, a portable computer, a thin client, or any suitable device or devices. The disclosure and/or components thereof may be a single device at a single location, or multiple devices at a single, or multiple, locations that are connected together using any appropriate communication protocols over any communication medium such as electric cable, fiber optic cable, or in a wireless manner.

It should also be noted that the disclosure is illustrated and discussed herein as having a plurality of modules which perform particular functions. It should be understood that these modules are merely schematically illustrated based on their function for clarity purposes only, and do not necessary represent specific hardware or software. In this regard, these modules may be hardware and/or software implemented to substantially perform the particular functions discussed. Moreover, the modules may be combined together within the disclosure, or divided into additional modules based on the particular function desired. Thus, the disclosure should not be construed to limit the present invention, but merely be understood to illustrate one example implementation thereof.

The computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Implementations of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

Implementations of the subject matter and the operations described in this specification can be implemented in digital electronic circuitry, or in computer software, firmware, or hardware, including the structures disclosed in this specification and their structural equivalents, or in combinations of one or more of them. Implementations of the subject matter described in this specification can be implemented as one or more computer programs, i.e., one or more modules of computer program instructions, encoded on computer storage medium for execution by, or to control the operation of, data processing apparatus. Alternatively or in addition, the program instructions can be encoded on an artificially-generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially-generated propagated signal. The computer storage medium can also be, or be included in, one or more separate physical components or media (e.g., multiple CDs, disks, or other storage devices).

The operations described in this specification can be implemented as operations performed by a “data processing apparatus” on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

### CONCLUSION

The various methods and techniques described above provide a number of ways to carry out the invention. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Certain embodiments of this application are described herein. Variations on those embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

Particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

What is claimed is:

1. A method for reducing hypoxia and hypercapnia during respiration of a patient, the method comprising:
providing a mechanical stimulator in proximity to the patient; and
applying a mechanical stimulation to the patient using the mechanical stimulator to reduce an amount of hypoxia experienced by the patient.

2. The method of claim 1 wherein the mechanical stimulation is applied on a continuous basis.

3. The method of claim 1 wherein the mechanical stimulation is applied on a periodic basis.

4. The method of claim 1 wherein the mechanical stimulation is automatically adjusted based on feedback from a respiration sensor.

5. The method of claim 4 wherein the adjustment is one of a magnitude, frequency, or duration of mechanical stimulation.

6. The method of claim 1 wherein the mechanical stimulation is automatically adjusted based on a measurement of respiratory function used to predict an upcoming respiratory state.

7. The method of claim 1 wherein the mechanical stimulation is applied for 30 minute on-and-off intervals.

8. The method of claim 1 wherein the mechanical stimulation has a frequency between 30-60 Hz.

9. The method of claim 1 wherein the mechanical stimulation has an intensity of 12 um+/−10% RMS.

10. The method of claim 1 wherein the patient is dependent on a ventilator.

11. The method of claim 1 wherein the patient is a healthy infant.

12. The method of claim 1 wherein the mechanical stimulator comprises at least one of the following: a speaker, a piezoelectric stimulator, a pulsed air mechanism, a hydraulic mechanism, or an electromagnetic actuator.

13. The method of claim 1 wherein the mechanical stimulation is applied to the patient's thorax.

14. The method of claim 1 further comprising:
receiving data output from a blood oxygenation sensor related to the patient;
analyzing the data to determine a blood oxygenation level; and
modifying a frequency of the mechanical stimulation in response to the blood oxygenation level.

15. The method of claim 1 further comprising:
receiving physiological data from the patient;
analyzing the physiological data to determine a weight of the patient; and
modifying a frequency of the mechanical stimulation in response to the weight of the patient.

16. A system for improving the respiratory function of a patient, the system comprising:
a mattress comprising an active zone;
a mechanical stimulator connected to the active zone;
a memory containing machine readable medium comprising machine executable code having stored thereon instructions for performing a method controlling the mechanical stimulator;
a processor coupled to the memory, the processor configured to execute the machine executable code to cause the processor to:
receive weight data regarding the patient;
determine an appropriate mechanical stimulus based on the weight data; and
apply the appropriate mechanical stimulus to the patient using the mechanical stimulator to improve respiratory function of the patient.

17. A method for reducing hypoxia during respiration of a patient, the method comprising:
providing a mechanical stimulator in proximity to the patient;
receiving physiological data output from a sensor from the patient;
determining a modified stimulus based on the physiological data; and
applying the modified stimulus to the patient using the mechanical stimulator to reduce an amount of hypoxia experienced by the patient.

18. The method of claim 17 wherein the physiological data is a blood oxygen saturation trend.

19. The method of claim 18 wherein the determining a modified stimulus comprises determining a new frequency of the modified stimulus based on the blood oxygen saturation trend.

20. The method of claim 19 wherein determining a new frequency of the stimulus based on the blood oxygen saturation trend comprises determining whether the blood oxygen saturation trend will likely cross below a threshold saturation.

21. The method of claim 20 wherein the threshold is 85 percent.

22. A system for improving the respiratory function of a patient, the system comprising:
a pressure support system configured to provide breathable air to the patient; and
a controller, wherein the controller is configured to:
receive a mechanical stimulation signal;
determining an altered signal based on deriving a set of parameters from the received mechanical stimulation signal; and
adjust an air pressure of the pressure support system based on the altered signal.

23. The system of claim 22, wherein the mechanical stimulation signal is automatically adjusted based on a measurement of respiratory function used to predict an upcoming respiratory state.

24. The system of claim 22, wherein determining the altered signal further comprising accounting for an optimal oxygen saturation of the patient.

25. The system of claim 22, wherein the adjusting is coordinated with the mechanical stimulation signal.

26. The system of claim 22, wherein the system further comprises a mechanical stimulator, wherein the mechanical stimulator is provided in proximity to the patient and is configured to:
receive the mechanical stimulation signal; and
mechanically stimulate the patient in response to receiving the mechanical stimulation signal.
